SEQUENCE LISTING (29 sequences)

SEQ ID NO: 1 - DNA, 39 bp, Artificial
  a forward primer; nucleotides 1 - 20 at 5′ end are T7 promoter sequences;  remaining nucloetides derived from human K-ras oncogene.
taatacgactcactatagggcctgctgaaaatgactgaa

SEQ ID NO: 2 - DNA, 20 bp, Artificial
  a reverse primer;  derived from human Kras oncogene
tactaggaccataggtacat

SEQ ID NO: 3 - DNA, 17 bp, Artificial
  artificial sequence, a forward primer
cctgcaggagacggtga

SEQ ID NO: 4 - DNA, 18 bp, Artificial
  artificial sequence, a reverse primer
tcaccgtctcctgcaggt

SEQ ID NO: 5 - DNA, 16 bp, Artificial
  artifical sequence,  a forward primer
gagcgagcagcagctg

SEQ ID NO: 6 - DNA, 17 bp, Artificial
  artificial sequence, a reverse primer
cagctgctgctcgctct

SEQ ID NO: 7 - DNA, 19 bp, Artificial
  a forward primer; sequence derived from human K-ras oncogene
tggtagttggagcttgtgg

SEQ ID NO: 8 - DNA, 19 bp, Artificial
  a forward primer; sequence derived from human K-ras oncogene
taggcaagagtgccttgac

SEQ ID NO: 9 - DNA, 19 bp, Artificial
  a forward primer; sequences derived from the second exon of BCR
cattccgctgaccatcaat

SEQ ID NO: 10 - DNA, 20 bp, Artificial
  a reverse primer; sequences derived from the seventh exon of the c-abl gene
acgtcggacttgatggagaa

SEQ ID NO: 11 - DNA, 40 bp, Artificial
  a forward primer derived from the third exon of the c-abl gene with T7 promoter sequences attached to its 5′ end
taatacgactcactatagggatcattcaacggtggccgac

SEQ ID NO: 12 - DNA, 17 bp, Artificial
  a forward primer; artificial sequence
gtcctcatgtactggtc

SEQ ID NO: 13 - DNA, 18 bp, Artificial
  a reverse primer; artificial sequence
gaccagtacatgaggact

SEQ ID NO: 14 - DNA, 20 bp, Artificial
  a forward primer; sequences derived fromTK domain of c-abl
gagaaccacttggtgaaggt

SEQ ID NO: 15 - DNA, 19 bp, Artificial
  a forward primer; sequences derived from TK domain of c-abl
tgagcaggttgatgacagg

SEQ ID NO: 16 - DNA, 20 bp, Artificial
  a reverse primer; sequences derived from the seventh exon of the c-abl gene plus an embedded BamHI site
taggggaacttggatccagc

SEQ ID NO: 17 - DNA, 25 bp, Artificial
  a forward primer; artificial sequences with an embedded Bgl II site
gagatcttgctgcccgaaactgcct

SEQ ID NO: 18 - DNA, 25 bp, Artificial
  a reverse primer; artificial sequences with an embedded Bgl II site
aggcagtttcgggcagcaagatctc

SEQ ID NO: 19 - DNA, 23 bp, Artificial
  a reverse primer; shares the same sequence as SEQ. ID. NO. 18 except it is shorter by two nucleotides at the 3′ end
aggcagtttcgggcagcaagatc

SEQ ID NO: 20 - DNA, 20 bp, Artificial
  a forward primer; sequences derived from TK domain of c-abl with a 6-FAM dye at 5 end, and a TAMRA dye at 3′ end.
gcatgggctgtgtaggtgtc

SEQ ID NO: 21 - DNA, 35 bp, Artificial
  a forward primer; sequences derived from the framework III of the V regions of the Ig heavy chain (IgH) gene and carries a T7 promoter sequence at its 5′ end
ttaatacgactcactatacggccgtatattactgt

SEQ ID NO: 22 - DNA, 20 bp, Artificial
  a reverse primer; sequences derived from the consensus sequences at the 3′ end of the JH region with an embedded Pst-1 restriction enzyme site
acctgcaggagacggtgacc

SEQ ID NO: 23 - DNA, 19 bp, Artificial
  a reverse primer; sequences derived from CDRIII region on monoclonal B-cells
agatattcatcaggtggtc

SEQ ID NO: 24 - DNA, 18 bp, Artificial
  a forward primer; sequences derived from T7 promoter
ttaatacgactcactata

SEQ ID NO: 25 - DNA, 19 bp, Artificial
  a reverser primer; sequences derived from CDRIII region on monoclonal B-cells
actggtgtggttcttggac

SEQ ID NO: 26 - DNA, 21 bp, Artificial
  a forward primer; sequences derived from cyclophilin
tgagacagcagatagagccaa

SEQ ID NO: 27 - DNA, 22 bp, Artificial
  a reverse primer; sequences derived from cyclophilin
tccctgccaatttgacatcttc

SEQ ID NO: 28 - DNA, 20 bp, Artificial
  a forward primer; sequences derived from CDRIII region on monoclonal B-cells  6-FAM dry at 5′ end, TAMRA dye at 3′ end
tgaactgggctgccgaggtg

SEQ ID NO: 29 - DNA, 38 bp, Artificial
  a reverse primer; sequences derived from cyclophilin with a VIC dye at 5′ end, TAMRA dye at 3′ end
agcaccaatattcagtacacagcttaaagctataggtt

(19) United States
(12) Patent Application Publication
(10) Pub. No.: US 2008/0318215 A1
(43) Pub. Date: Dec. 25, 2008

(54) APPARATUS, METHODS AND PRODUCTS FOR DETECTING GENETIC MUTATION

(76) Inventors: Ming-Sheng Lee, Sugarland, TX (US); Chung-Han Lee, Sugarland, TX (US)

Correspondence Address: J. M. (Mark) Gilbreth;GILBRETH ROEBUCK, P. O. Box 2428, Bellaire, TX, 77402-2428, US

(21) Appl. No.: 11/614,041

(22) Filed: Dec. 20, 2006

Related U.S. Application Data
(60) Provisional application No. 60/752,122, filed on Dec. 20, 2005.

(51) Int. Cl. C12Q 1/68; C12M 1/00

(52) U.S. Cl. 435/6; 435/287.2

(57) ABSTRACT

Methods for detecting genetic mutation allowing detection of very low frequency mutation. Methods comprise treating RNA:DNA heteroduplexes of interest with ribonuclease treatment coupled with DNA polymerase treatment. RNA:DNA heteroduplexes of interest are preferentially targeted for digestion by ribonuclease and subsequent sequence extension by DNA polymerase. Methods may be carried out partially or entirely manually, automatically, and combinations thereof. Methods may be performed wholly or partially in solution, on solid phase media, in large scale, adapted for high throughput analysis, and any combinations thereof. Apparatus and products for detecting genetic mutation.

### CROSS REFERENCE TO RELATED APPLICATION

The present application claims priority to U.S. Provisional Patent Application Ser. No. 60/752,122, entitled “Sensitive Detection of Genetic Mutations through Differential Sequence Extension (DSE)-mediated Ligation Followed by Nucleic Acid Sequence Amplification and High Throughput Genetic Characterization through Differential Sequence Blockage (DSB) and DSE-mediated Ligation, filed Dec. 20, 2005, which is hereby incorporated by reference.

### BACKGROUND

1. Technical Field

The present disclosure relates to apparatus, methods and products in the field of detection and analysis of genetic mutation.

2. Background Information

Genetic alterations play a role in a vast array of diseases and medical conditions. For example, genetic alterations are involved in the numerous phases of cancer progression including initial mutational events, benign and malignant cellular transformation, development of metastasis, and even the development of resistance to therapy. It remains a great challenge for clinicians and researchers to detect minimal residual disease in patients in clinical remission, or to identify individuals who appear healthy clinically, but harbor a very small number of mutant cells that are at risk for developing into malignant tumors.

Numerous assays for the detection of genetic mutations are available for clinical and research applications, for example but not limited to the Single-Stranded DNA Conformation Polymorphism (SSCP) assay, the heteroduplex formation assay, high performance liquid chromatography (HPLC)-based mutation screening by WAVE technology, the ribonuclease protection assay, the dot blots or reverse dot blots analysis, and DNA sequencing. Despite the large number of assays available to researchers and clinicians, the detection sensitivities of these assays are limited to approximately 10% and lower. Therefore, it remains a daunting challenge to attempt to detect small numbers of mutants among hundreds of thousands of normal cells using current technologies.

Efficiency is another area of limitation with conventional technologies. Although recent advances in microarray technology facilitate simultaneous examination of large numbers of different genes for differential gene expression, they do not allow for screening large numbers, for example, thousands, of different genes for the presence of a single mutation. For example, the detection of an abnormality residing at a mutation hot spot in a particular target gene, or the molecular identification of tumor suppressor genes, would require hundreds of different probes carrying various mutant sequences representing all possible mutations. In order to screen thousands of different genes, each with different mutation hot spots, or to search for an unknown mutation in a genetic region of interest, upwards of billions of different probes would be necessary. Conventional technologies also lack the ability to provide in situ characterization of genetic mutations while preserving cell morphologies of tissue sections or cell preparations on slides for observation such as with a microscope.

The methods, apparatus and products of the present disclosure overcome these and additional deficiencies of conventional technology and enable sensitive detection of a mutant present in a normal background population at a very low frequency.

### SUMMARY

The following presents a general summary of some of the many possible embodiments of this disclosure in order to provide a basic understanding of this disclosure. This summary is not an extensive overview of all embodiments of the disclosure. This summary is not intended to identify key or critical elements of the disclosure or to delineate or otherwise limit the scope of the claims. The following summary merely presents some concepts of the disclosure in a general form as a prelude to the more detailed description that follows.

According to one non-limiting embodiment there is provided a method for detecting a genetic alteration. Generally the method comprises the step of incubating at least one sample of heteroduplex molecules comprising a genetic region of interest with a ribonuclease enzyme, wherein the heteroduplex molecules comprise one strand of sense ribonucleic acid (RNA) and one strand of antisense deoxyribonucleic acid (DNA). The sample of heteroduplex molecules comprises a first population of heteroduplexes wherein the RNA and DNA strands are fully hybridized to one another, and a second population of heteroduplexes having at least one member, wherein the RNA strand of the at least one member comprises at least one unhybridized nucleotide within the region of interest, wherein the ribonuclease cleaves 3′ of said unhybridized nucleotide exposing a 3′ hydroxyl group. The at least one unhybridized nucleotide generally corresponds to a mutation. The method also comprises the steps of synthesizing a strand of DNA from the 3′ hydroxyl group wherein the antisense DNA is used as a template to produce a sequence extended heteroduplex; linking a marker to the sequence extended heteroduplex to form a marked heteroduplex; and detecting the marked heteroduplex.

According to another non-limiting embodiment there is provided a method for detecting a genetic mutation. Generally the method comprises the step of incubating at least one sample of single stranded RNA together with at least one sample of single stranded antisense DNA to create at least one sample of RNA:DNA heteroduplex molecules comprising a region of interest. Generally the at least one sample of single stranded antisense DNA is immobilized on a substrate, and the resulting sample of RNA:DNA heteroduplexes is immobilized. The sample of RNA:DNA heteroduplexes comprises a first population of heteroduplexes wherein the RNA and DNA strands are fully hybridized to one another, and a second population of heteroduplexes having at least one member, wherein the RNA strand of the at least one member comprises at least one unhybridized nucleotide within the region of interest. The method also comprises the steps of: incubating the sample of RNA:DNA heteroduplex molecules with a ribonuclease enzyme wherein the ribonuclease cleaves 3′ of the unhybridized nucleotide exposing a 3′ hydroxyl group; synthesizing a strand of DNA from the 3′ hydroxyl group wherein the antisense DNA is used as a template to produce a sequence extended heteroduplex; linking a marker to the sequence extended heteroduplex to form a marked heteroduplex; and detecting the marked heteroduplex.

According to another non-limiting embodiment there is provided a kit for detecting genetic mutation. Generally the kit comprises a multitude of single stranded antisense DNA probes immobilized on a substrate, wherein each of said DNA probes comprises a unique genetic region of interest, and wherein each of said DNA probes is located at a unique location on said substrate; and a user's guide comprising instructions for executing a method of the present disclosure.

According to another non-limiting embodiment there is provided an apparatus for detecting genetic mutation. Generally the apparatus comprises a reaction chamber comprising at least one removable sample holding device, four walls, a ceiling and a floor, wherein one of the walls comprises a door. The apparatus further comprises a temperature control element is positioned within the reaction chamber and regulated the temperature of reaction conditions within the chamber. The apparatus further comprises an electromagnetic member that can be turned on to induce magnetism and turned off to remove magnetism is positioned within the reaction chamber. The apparatus further comprises a fluid-dispensing element for adding and removing reaction materials to samples when samples are present in the reaction chamber is positioned in such a way as to have access to the reaction chamber. Each of the temperature control element, electromagnetic member, and fluid dispensing element are movable and may be repositioned to be in proximity with samples when samples are present in the reaction chamber. The apparatus further comprises a fluorometer coupled with the reaction chamber that may be used to detect any fluorescence present in the chamber.

### BRIEF DESCRIPTION OF THE DRAWINGS

The following drawings illustrate some of the many possible embodiments of this disclosure in order to provide a basic understanding of this disclosure. These drawings do not provide an extensive overview of all embodiments of this disclosure. These drawings are not intended to identify key or critical elements of the disclosure or to delineate or otherwise limit the scope of the claims. The following drawings merely present some concepts of the disclosure in a general form. Thus, for a detailed understanding of this disclosure, reference should be made to the following detailed description, taken in conjunction with the accompanying drawings, in which like elements have been given like numerals.

FIG. 1 illustrates a non-limiting approach for reducing background.

FIG. 2 illustrates a non-limiting approach for reducing background.

FIG. 3 provides an overview of a non-limiting method for detecting genetic mutation comprising a wild type probe.

FIG. 4 provides an overview of a non-limiting method for detecting genetic mutation comprising a mutant probe.

FIG. 5 provides an overview of a non-limiting method for detecting genetic mutation comprising two species of adapters.

FIG. 6 provides an overview of a non-limiting method for detecting multiple genetic mutations simultaneously in microarray format.

FIG. 7 provides an overview of a non-limiting method for detecting genetic mutation comprising in situ transcripts.

FIG. 8 provides an overview of a non-limiting method for detecting genetic mutation comprising solid-phase media.

FIG. 9 provides an overview of a non-limiting method for detecting genetic mutation comprising magnetic solid-phase media

FIG. 10 is a schematic of a non-limiting apparatus of the disclosure.

FIG. 11 is a schematic of a non-limiting apparatus of the disclosure.

FIG. 12 shows the products of a method of the disclosure for detecting Ras mutations.

FIG. 13 is a schematic of a method of the disclosure for detecting Ras mutations.

FIG. 14 is a schematic of a method of the disclosure for detecting TK mutations.

FIGS. 15A and 15B show the products of a method of the disclosure for detecting TK mutations.

FIGS. 16A and 16B show the plots of real-time PCR on TK mutants.

FIGS. 17A and 17B provide schematics of a DSE assay for detecting B-cell mutations.

FIG. 18 shows the products of a method of the disclosure for detecting B-cell mutations.

FIG. 19 shows amplification plots of B-cell leukemia positive control cells.

FIGS. 20A and 20B show the amplification plots of remission bone marrow and peripheral blood stem cells.

FIG. 21 shows the products of a method of the disclosure for detecting B-cell mutations.

FIG. 22 is a schematic of a method of the disclosure for detecting microsatellite polymorphisms.

FIG. 23 depicts real-time PCR plots of microsatellite polymorphism mutation analysis.

FIG. 24 shows the products of a method of the disclosure for detecting microsatellite polymorphisms.

FIGS. 25A and 25B provide amplification plots of the products of FIG. 23.

FIG. 26 shows the amplification plots of TK mutants identified by a solid phase media DSE assay.

### DETAILED DESCRIPTION

The apparatus, methods and products of the present disclosure enable the detection of one or more genetic mutations. As used herein, the term “genetic mutation” includes all genetic alterations known in the art, for example but not limited to, point mutations, nucleotide polymorphisms, deletions, insertions, microsatellite instabilities, translocations, and all combinations thereof. The genetic mutation may comprise one or more genetic alterations and these may be the same or different types of alterations. The genetic mutation may be in any region of any genetic material, such as within and/or near any one or more gene, and may be within and/or near any coding or noncoding region. The genetic material may be deoxyribonucleic acid (DNA), ribonucleic acid (RNA), or both, and may be any size. Though not a requirement for the genetic mutations of the present disclosure, the mutation may be associated, directly or indirectly, with a medical condition for example, a cancer. In one non-limiting embodiment, the mutation is associated with any stage of initialization, development, progression, and/or remission of any cancer. The mutation may be a marker for a disease. The mutation may be associated with a resistance to therapy, for example resistance to any pharmaceutical compound or drug, resistance to any type of chemotherapy, resistance to any type of radiotherapy, and any combinations thereof. The mutation may be a nucleotide sequence variation among normal individuals, or single nucleotide polymorphism (SNP). SNPs are widely used in assessing chimerism post allogeneic blood or bone marrow stem cell transplantation, and also for detecting loss of heterozygosity (LOH) as an evidence of chromosomal deletion. Thus, the apparatus, methods and products of the present disclosure may be utilized for any one or more of these situations but are by no means limited to these situations.

Nucleic acid from any source and/or sample may be used in the apparatus, methods and products of the disclosure. The sample may be isolated from any one or more individuals and/or organisms. The sample may be any cell, tissue or fluid sample, including but not limited to, skin, plasma, serum, spinal fluid, lymph fluid, synovial fluid, urine, tears, blood cells, organs, tumors, any biopsy sample, a tissue section sample, a cell preparation sample, and any cell culture sample including in vitro cell culture constituents, such as but not limited to conditioned medium resulting from the growth of cells in cell culture medium, recombinant cells and cell components. The nucleic acid may also be derived from any molecular biology, microbiology, and/or recombinant DNA technique known in the art.

The apparatus, methods and products of the present disclosure enable detection of a genetic mutation, which may be present in a sample at a very low frequency. For example, the mutant may be present among several hundred thousands of non-mutants. In contrast to conventional mutation detection assays which are limited to detection sensitivities of approximately less than 10%, the apparatus, methods and products of the present disclosure provide detection sensitivity of generally from ten-fold to a hundred-fold higher, and in some cases, several thousand-fold higher, than that of conventional technology. The apparatus, methods and products of the present disclosure enable mutation analysis of any one or more genetic region of interest, in any one or more sample which may be analyzed simultaneously, or sequentially, or any combinations thereof.

One characteristic feature of the apparatus, methods, and products of the pre sent disclosure is the coupling of an RNase treatment step with an RNA-primed DNA polymerase treatment step enabling selective sequence extension of genetic regions of interest. Generally the methods of the disclosure comprise incubating a sample of RNA:DNA heteroduplexes of interest with a ribonuclease enzyme (RNase) under reaction conditions suitable for ribonuclease activity. The RNase may be any of the RNases known in the art but is generally an RNase having endonuclease activity (i.e., an endo-ribonuclease). Preferably the RNase cleaves 3′ of the substrate ribonucleotide base exposing a free 3′ hydroxyl (OH) group. The RNA:DNA heteroduplexes useful herein comprise a single strand of sense RNA and a single strand of antisense DNA wherein the strands are approximately the same length. Generally within the sample population of RNA:DNA heteroduplex molecules there will be fully matched/annealed heteroduplex molecules wherein the sequences of the single stranded RNA are completely complementary to those of the antisense DNA. Within the sample of RNA:DNA heteroduplexes, there may also be partially mismatched heteroduplex molecules wherein the sequences of the single stranded RNA and the antisense DNA have at least one nucleotide difference from one another and thus are not completely complementary sequences. For those partially mismatched RNA:DNA heteroduplex molecules having mismatched ribonucleotides that did not undergo base-pairing, incubation with ribonuclease will result in digestion of the mismatched ribonucleotides producing a nick in the RNA strand at the site of the mismatched ribonucleotides. In contrast, the fully matched/annealed heteroduplexes will be immune from ribonuclease activity since they lack the appropriate target substrate, i.e., a mismatched/unhybridized ribonucleotide.

The RNase treatment of the RNA:DNA heteroduplexes is coupled with a step referred to herein as “differential sequence extension” (“DSE”) wherein an RNA-primed DNA polymerase is incubated with the RNase treated, RNA:DNA heteroduplexes under reaction conditions suitable for DNA polymerase activity. The RNA-primed DNA polymerase may be any such polymerase known in the art including, but not limited to, Klenow fragments of E. coli DNA polymerase, Klenow Exo⁻ (a variant of Kienow enzyme that lacks 3′ to 5′ exonuclease activity), or sequential utilization of a Klenow enzyme followed by Taq DNA polymerase. The RNA-primed DNA polymerase is able to utilize the nicked RNA fragments of the nicked heteroduplexes as a primer, and will synthesize/extend a new DNA strand using the complementary DNA strand of the heteroduplex as a template. The nicked fragments within the nicked heteroduplexes are suitable primers for the RNA-primed DNA polymerase because of the free hydroxyl (OH) group at their 3′ end exposed as a result of ribonuclease digestion. In contrast, the RNA-primed DNA polymerase has no activity on the intact, fully hybridized heteroduplexes and thus the name “differential sequence extension” for this step.

Generally it is preferred that the DSE heteroduplexes have sticky ends in order to facilitate any subsequent ligation reactions. The type of sticky end produced on the DSE heteroduplex molecules depends on the RNA-primed DNA polymerase selected for use in the DSE step, and whether any modifications are carried out on the heteroduplex. For example, the use of Klenow exo- and ddNTP blockage on a 5′ dinucleotide “AA” overhang embedded on the antisense, wild type DNA probe results in a single nucleotide “ddT” sequence extension on the RNA strand, thus creating a 3′ single nucleotide recessive end. With respect to the complementary DNA strand, this is viewed as a 5′ single nucleotide “deoxyA” overhang. As another example, performing DSE with sequential utilization of Klenow enzyme and Taq DNA polymerase results in a 3′ single nucleotide “deoxy-A” overhang because of the terminal deoxynucleotide transferase (TdT) activity of Taq DNA polymerase. The TdT activity of Taq DNA polymerase results in a preferential incorporation of a “deoxy-A” at the 3′ end of the extended sequence without the need of a DNA template. Generally, when using these two polymerase sequentially, following priming by Klenow, the possessiveness of Taq DNA polymerase enhances sequence extension for up to as many as a several kilobases (kbs), thus permitting a much longer range of sequence extension than the use of Kienow or Klenow exo⁻ alone. Performing the DSE step with sequential utilization of Klenow enzyme and Taq DNA polymerase is a preferred approach for the DSE, especially when the sequence extension is great than about 400 to 500 nucleotides. If it is known that short extensions of less than about 400 to 500 nucleotides are needed, use of Klenow alone may be sufficient. In those situations where the length of sequence extension is unknown, use of sequential Klenow/Taq may be preferred in order to ensure complete extension in the DSE step.

Performing DSE with Klenow alone results in blunt ends that can be overcome by embedding a restriction enzyme (“Res”) sequence near the 3′ end of the antisense DNA probes during synthesis of the probe. The resulting DSE heteroduplex created from this “Res” containing template DNA probe during the DSE step will contain a “Res” site. “Res” digestion of this “Res” containing DSE heteroduplex will then reveal the “Res” sticky ends. This approach for creating a restriction enzyme site is known by those of skill in the art. Incorporation of a restriction enzyme site into the DNA probe is useful when DSE is to be performed with sequential utilization of Klenow enzyme and Taq DNA polymerase.

Using standard techniques of recombinant and molecular biology, adapters with sticky ends complementary to those of the DSE heteroduplexes may then be ligated to the DSE heteroduplexes. Conditions and techniques for creating adapters are well known in the art and all are suitable for use herein. Ligation conditions and techniques are also known in the art and all are suitable for use herein. Ligation of a unique adapter to the DSE heteroduplex allows for detecting and quantitating the adapter-heteroduplex. Because the fully matched heteroduplexes are blunt-ended and the adapters have sticky ends, ligation does not generally occur between them. Generally the adapters are at least 10 base pairs (bp) in length, preferably at least 12 bp, and may be as large as several hundred bp. In one non-limiting embodiment the adapters are from about 18 to about 30 bp in length.

Following ligation, the adapter-heteroduplex hybrids may be detected and/or quantitated by any applicable method known in the art. One non-limiting approach is to use PCR to preferentially amplify the adapter-heteroduplexes, which may be achieved by use of PCR wherein one primer of the primer pair used for PCR is specific to sequences within the adapter and the other primer is specific for the target gene of interest. The ability to selectively amplify the adapter-heteroduplexes enables sensitive detection and quantification of a small number of targets of interest in a sample of hundreds of thousands of non-targets.

The adapter molecules may comprise or be coupled with a tag useful in detection and/or quantitation of the adapter-heteroduplex molecules. As used herein a “tag” refers to any atom or molecule which may be used to confer a detectable and/or quantifiable signal, and which may be attached to a nucleic acid or protein. Any tag known in the art may be utilized herein including, but not limited to, a fluorescent dye, a colorization agent, a radioactive isotope, a chemiluminescent substrate, a luciferase substrate, a magnetic tag/bead, and all combinations thereof. The tag may be detectable and/or quantifiable by any suitable technique known in the art including, but not limited to, fluorimetry colorimetry, scintillation counting, autoradiography, use of any type of camera such as a CCD camera, luminometry, magnetism, enzymatic activity, gravimetry, X-ray diffraction or absorption, and all combinations thereof.

Following the ligation of adapter-heteroduplex hybrids, the ligation products may be treated with a nuclease specific for single stranded nucleic acid, such as but not limited to S1 nuclease, as an approach to eliminate single stranded DNA background molecules such as competitive templates and primers, which may decrease the efficiency of subsequent detection and/or quantitation steps, such as PCR. Treatment with a nuclease such as S1 nuclease will hydrolyze any single stranded DNAs present in the ligation mixture regardless of whether they are free DNAs or have undergone non-specific ligation to an adapter. Following S1 nuclease digestion and prior to performing PCR, any purification method known in the art, for example, size-exclusion column purification, may be employed to remove oligonucleotides or nucleic acids that may otherwise competitively inhibit the subsequent PCR reaction.

The adapter heteroduplex molecules may also be used in any recombinant DNA and molecular biology technique useful for further characterization of the mutant sequence. For example, the sequence may be ligated to any eukaryotic and/or prokaryotic expression vector and/or plasmid for further analysis and characterization.

Any technique known in the art for creating heteroduplex molecules may be used herein to create the RNA:DNA heteroduplexes of interest. In one non-limiting embodiment, a sample of single stranded RNA comprising a genetic region of interest and a sample of single-stranded, antisense DNA (the probe) having sequences complementary to the RNA of interest are subject to reaction conditions suitable for nucleic acid hybridization, resulting in the formation of RNA:DNA heteroduplexes. The sample of single stranded RNA of interest may comprise a population of RNA having wild type/normal sequences and may also comprise a population of RNA having a mutant sequence. The sample of single stranded RNA of interest may comprise more than one population of RNAs having a mutant sequence wherein each of the more than one population has a mutation sequence unique to that population. Any population of RNA having a mutant sequence may comprise one member, or more than one member.

The antisense DNA probe may be complementary to the wild type sequences of interest, or may be complementary to a mutant sequence of interest. It should be clear to one of skill in the art that if the DNA probe is designed to comprise wild type sequences, hybridization of the wild type probe with complementary wild type RNAs will produce a fully matched RNA:DNA heteroduplex (i.e., no unhybridized/mismatched ribonucleotides), whereas hybridization of a wild type probe with RNAs containing a mutation will result in RNA:DNA heteroduplexes comprising partially mismatched, unhybridized ribonucleotides. Alternatively, if the antisense DNA probe is a mutant sequence, the probe will fully hybridize with RNAs carrying the same mutation thereby creating fully matched RNA:DNA heteroduplexes, but will be partially mismatched with wild type RNAs thereby creating partially mismatched RNA:DNA heteroduplexes having unhybridized ribonucleotides. Either a wild type DNA probe or a mutant DNA probe may be used in the apparatus, methods and products of the present disclosure.

The single stranded RNA of interest used to create the RNA:DNA heteroduplexes may be from any suitable source, or the product of any method known in the art for synthesis and/or amplification of RNA including, but not limited to, in vitro transcription, transcription-mediated amplification (TMA), and polymerase chain reaction (PCR) amplification coupled with in vitro transcription. The synthesis and/or amplification of RNA transcripts may target transcripts specific to a single genetic region of interest, or may target transcripts from more than one genetic region of interest, or may target total cellular RNA and/or transcripts.

In a non-limiting embodiment, amplification of transcripts is achieved by performing reverse transcription on a sample of RNAs comprising a region of interest. The resulting cDNAs may then be subject to any PCR amplification, including but not limited to nested and semi-nested PCR, in order to target and amplify the region of interest therein. As known in the art, any primer used for PCR may have embedded therein any one or more sequence useful for subsequent manipulation of the resulting amplicon and/or any product resulting from said amplicon. For example, the forward primer of the pair of primers used in the PCR reaction may comprise a promoter sequence for an RNA polymerase, such as but not limited to, a T7, T3, or SP6 RNA polymerase, thereby introducing the promoter sequence into the resulting amplicon allowing for subsequent transcription therefrom. Additional sequences which may be embedded in the primers used for PCR amplification which allow for subsequent manipulation of the resulting amplicons include, but are not limited to restriction enzyme sites, M13 sequences, and the like. Following PCR amplification, the amplicons may be purified by any suitable method, such as but not limited to, use of a QIAquick PCR purification kit (QIAGEN, Valencia, Calif.). The purified amplicons may then be subject to RNA transcription with the appropriate RNA polymerase, for example T7, T3, or SP6 RNA polymerase, in the appropriate reaction buffer. Following in vitro transcription, the resulting transcripts of interest may be treated with DNase to produce DNA-free, transcripts of interest. The DNA-free, single stranded RNA transcripts of interest may be denatured prior to hybridization in order to maximize efficiency of RNA:DNA heteroduplex formation. Generally the length of the transcripts used in creating the RNA:DNA heteroduplexes of the present disclosure is in the range of about 20 bases to several kilobases, preferably in the range of about 50 to about 1000 bases.

The single stranded antisense DNA probes useful in creating the RNA:DNA heteroduplexes may be the product of any method known in the art to be useful for producing single stranded antisense DNA, for example but not limited to, reverse transcription (RT) PCR, followed by PCR with a single primer. In one non-limiting embodiment, a sample of RNA comprising a region of interest is used as a template for RT-PCR resulting in amplification of a cDNA fragment of the region of interest. The cDNA may then be used as a template to synthesize an antisense, single-stranded DNA probe by performing PCR with a reverse primer. The methods of the disclosure allow for use of either DNA probes specific for wild type sequences, or DNA probes specific for mutant sequences. In order to create a probe having wild type sequences of interest, use of wild type RNA is used as the RNA template for RT-PCR. If a probe having mutant sequences is desired, the RNA template for RT-PCT is RNA carrying the mutation of interest. Generally the length of the probes used in creating the RNA:DNA heteroduplexes of the present disclosure is in the range of about 20 nucleotides to several kilobases, preferably in the range of about 50 to about 1000 bases.

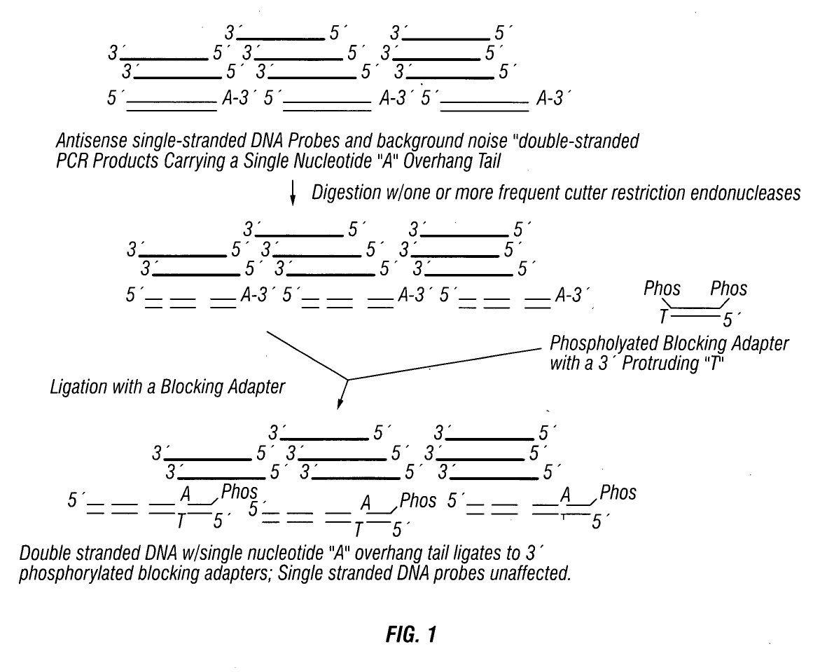

Following the preparation of the single-stranded antisense DNA probe by PCR, it is possible for there to be double stranded PCR products present in the reaction products. It is preferred that these double stranded DNAs be blocked and thus prevented from contributing background noise in the subsequent steps involving the probe. One useful blocking approach, outlined in FIG. 1, is to incubate the PCR products with one or more with frequent-cutter restriction enzymes such as, but not limited to, BamHI, HhaI, HpaII, HaeIII, MboI, and any combinations thereof, in order to digest any double stranded DNAs present therein. Being single-stranded, the antisense DNA probe is unaffected by the restriction enzymes. The sticky ends of the digested double stranded DNA fragments may then be blocked by incubating the products with Klenow exo⁻ and dideoxyribonucleotides (ddNTP's). This manipulation alters the sticky ends and prevents the ddNTP-blocked DNAs from undergoing non-specific sequence extension in the subsequent DSE step. Optionally the blocking step may be enhanced by adding terminal deoxyribonucleotide transferase (TdT), which incorporates ddNTP's randomly to the 3′ end of any double stranded DNA without the need of a template. The uncut single stranded DNA probe is not affected by any of these suggested blocking treatments except for attachment of an inert single nucleotide “ddNTP” at its 3′ end.

Following restriction enzyme digestion, blocking adapters having sticky ends complementary to the sticky ends of the digested DNA fragments may be ligated to the digested DNA fragments in order to block them from contributing background noise in the subsequent steps, for example the DSE step. The blocking adapter depicted in FIG. 1 comprises a single nucleotide “T” overhang on the 3′ end of its antisense strand. This “T” overhang is complementary to the single nucleotide “A” overhang present on any background double stranded DNA molecules and thus enables ligation between the blocking adapter and background molecule. The blocking adapter is also phosphorylated on both the 5′ and 3′ ends of its sense strand, as shown in FIG. 1. The 5′ and 3′ phosphorylations of the sense strand of the adapter respectively enable ligation of the adapter, and prevention of 3′ sequence extension in the DSE step. The use of blocking adapters is required and especially if the subsequent DSE step creates a 3′ single nucleotide “A” overhang on the DSE heteroduplexes. Though the adapter depicted in FIG. 1 is phosphorylated on the 3′ end of the sense strand, the blocking of this 3′ end may be achieved by any method that results in covalently bonding the 3′ OH group of the end with an inert group.

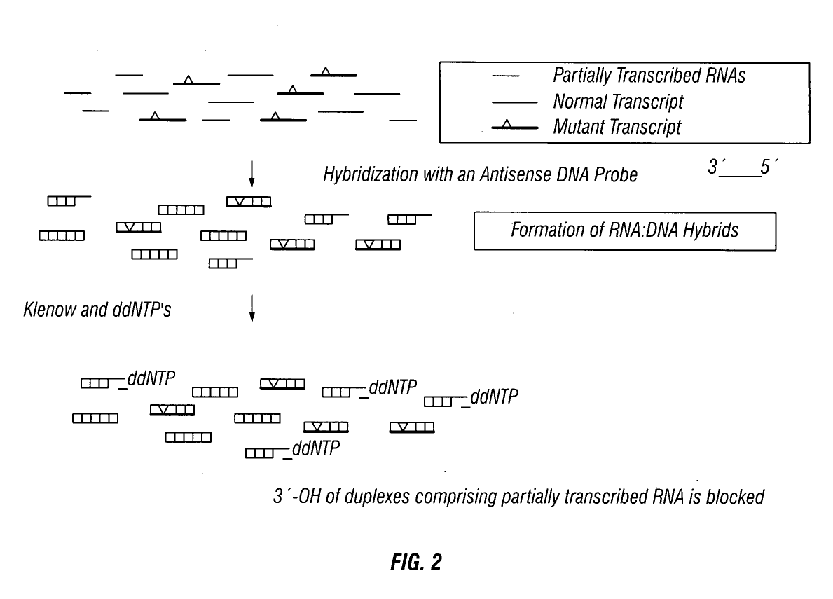

During hybridization of RNA:DNA heteroduplexes of interest, it is possible for there to be partially transcribed RNAs (“background noise” for example from transcript amplification) present in the reaction mixture. Hybridization between the DNA probe and these partially transcribed RNAs results in incomplete duplexes comprising a 3′ underhang with a free 3′ OH group. In order to block these 3′ OH groups of these “background noise” duplexes from undergoing undesired/nonspecific sequence extension during the DSE step, the hybridization products may be incubated together with Klenow enzyme and dideoxynucleotides (ddNTPs) under reaction conditions suitable for Klenow activity. This blocking approach is illustrated in FIG. 2.

The hybridization reactions carried out to create the RNA:DNA heteroduplex molecules utilized in the present disclosure may comprise any one or more DNA probe, wherein each probe is specific for a different genetic region of interest. For example, probe A specific for gene A, probe B specific for gene B, probe C specific for gene C. The hybridization reactions may comprise any one or more species of transcript, for example and keeping with the A, B, C nomenclature, transcript A of gene A, transcript B of gene B, transcript C of gene C, etc. Thus, the resulting RNA:DNA heteroduplexes may comprise more than one species/population of RNA:DNA heteroduplexes, for example, heteroduplexes A, heteroduplexes B, heteroduplexes C. Any one or more of these species of heteroduplexes may contain one or mutant members, for example, mutant A, mutant B, mutant C. It is also possible that more than one type/species of mutation is present and detected. For example, mutant A1 having mutation 1, mutant A2 having mutation 2, mutant A3 mutation 3. Each different mutation may be represented/populated by any one or individual members.

The mutation detection methods in the present disclosure comprise a series of different reactions, wherein each reaction comprises reaction products and/or reagents. The products and reagents of one step may interfere with the subsequent reaction(s). During the development and creation of the methods of the disclosure, it has been discovered that in order to achieve highly sensitive detection assays, reduce the background noise, and optimize the many different reactions of the methods of the disclosure, the following manipulations are required: i) the DNase treatment step after in vitro transcription during preparation of the single stranded RNA transcripts used to create the RNA:DNA heteroduplex samples; ii) digestion with frequent cutter restriction enzymes and subsequent blocking with use of Klenow or Klenow exo- and ddNTPs following synthesis of antisense single-stranded DNA probe by PCR; iii) the use of blocking adapters and especially in situations wherein A/T ligation is to be performed after DSE; and iv) blocking by use of Klenow or Klenow exo⁻ enzyme and ddNTPs after probe hybridization and formation of RNA:DNA heteroduplexes prior to performing ribonuclease digestion. The additional blocking step comprising use of TdT and ddNTP to block any remaining free 3′-OH groups prior to performing ribonuclease digestion is optional. The S1 nuclease treatment step following the ligation of adapters to the DSE heteroduplexes/products is optional.

With respect to product purification, performing purification after steps involving ddNTP blockage and after the use of blocking adapters is required. Any applicable purification method known in the art may be carried out such as, but not limited to, the use of a size exclusion column, for example a Quiaquick PCR purification column or a G50 Sephadex column. The exclusion limit of these columns is typically about 100 bps in length, thus ideal for removing ddNTP's, blocking adapters, and short nucleic acids less than 100 bps. Product purification in solid-phase media-immobilized DSE assays may be easily accomplished by washing with any one or more washing buffers.

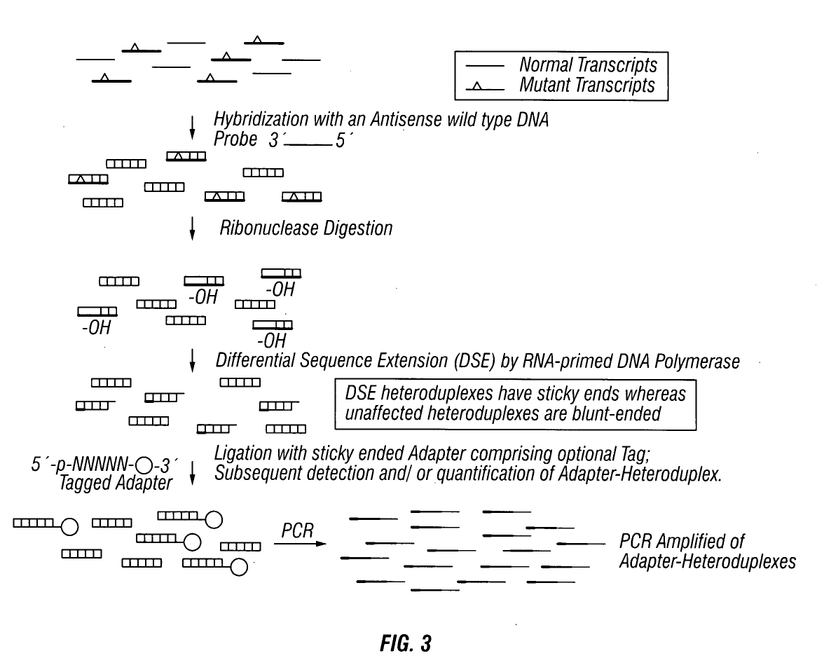

Referring now to FIG. 3 there is provided an overview of a non-limiting method for detecting a mutation in a genetic region of interest. As shown in FIG. 3, a sample comprising normal (wild type) single stranded RNA transcripts of interest and mutant RNA transcripts of interest (indicated by a filled triangle) are subject to hybridization with a single stranded antisense DNA probe to create RNA:DNA heteroduplex molecules. In this non-limiting embodiment, the antisense probe is complementary to wild type DNA sequences and thus fully hybridizes with the wild type transcripts to produce fully matched RNA:DNA heteroduplexes. In contrast, hybridization of the wild type probe with the mutant transcripts results in mismatched ribonucleotides (indicated by a filled triangle) in the RNA:DNA heteroduplexes. The RNA:DNA heteroduplexes are subjected to ribonuclease digestion that nicks the partially mismatched heteroduplexes leaving a free 3′ OH group on the nicked fragment. This nicked fragment serves as a primer in the subsequent DSE step. As indicated, the RNA-primed DNA polymerase leaves a sticky end on the DSE heteroduplexes while the unaffected fully matched heteroduplexes remain blunt-ended. Taking advantage of the sticky ends of the DSE heteroduplexes, adapters with complementary sticky ends may be ligated thereto. The resulting adapter-heteroduplex molecules may then be detected and/or quantified, for example by using PCR. If the adapter comprises a tag, the tag may also be used for detection and/or quantitation of the adapter-heteroduplex molecules.

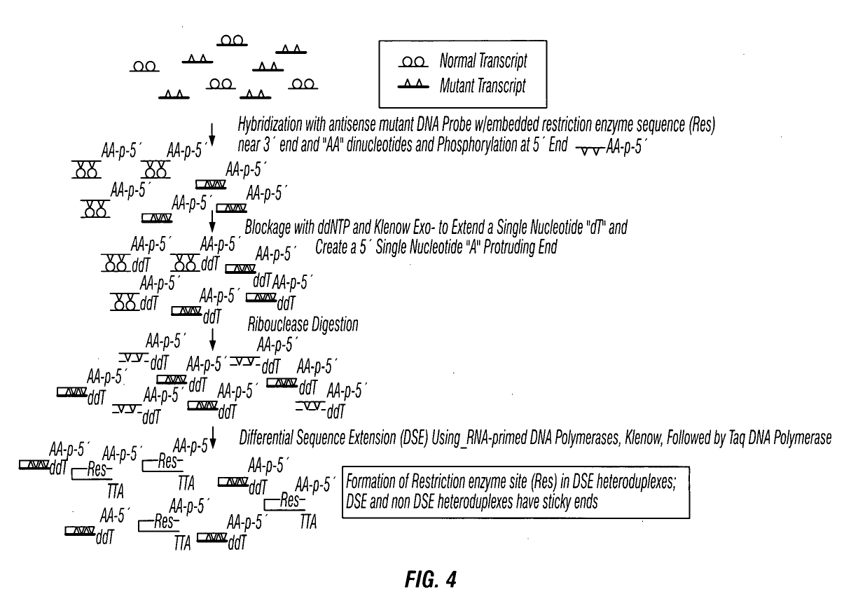

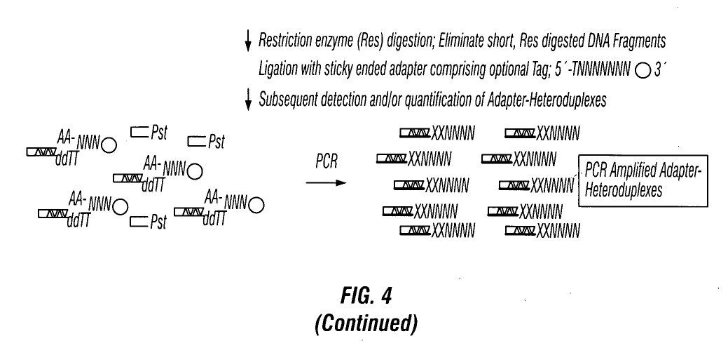

Referring now to FIG. 4 there is provided an overview of a non-limiting method for detecting genetic mutation comprising use of a probe carrying a known mutation of interest. As indicated, a sample comprising normal RNA transcripts of interest and mutant RNA transcripts of interest (indicated by a filled triangle) are subject to hybridization with a single stranded antisense DNA probe. In this non-limiting embodiment, the antisense probe is complementary to mutant DNA sequences and thus fully hybridizes with the mutant transcripts to produce fully matched RNA:DNA heteroduplexes but results in partially mismatched RNA:DNA heteroduplexes upon hybridization with the wild type transcripts. In this non-limiting embodiment, the probe also comprises a restriction enzyme site (“Res”) embedded near its 3′ end (not shown), and the dinucleotide “AA” and phosphorylation at its 5′ end. These modifications will aid in the subsequent manipulation of the RNA:DNA heteroduplexes. Any restriction enzyme sequence may be utilized for example, but not limited to, PstI, or BamHI, and preferably one that is unique to either the fully matched RNA:DNA heteroduplexes or the DSE heteroduplexes to allow for specific restriction enzyme digestion of one species of heteroduplex or the other. Following RNA:DNA heteroduplex formation and prior to the RNase treatment and DSE step, differential sequence blocking (DSB) is carried out using an RNA-primed DNA polymerase, such as Klenow exo⁻, and ddNTPs. As a result, a single nucleotide “ddTTP” is extended from the RNA strand. The 5′ dinucleotide “AA” protruding end of the RNA:DNA heteroduplexes is thus changed into a 5′ single nucleotide “A” overhang. Following ribonuclease digestion and subsequent DSE by sequential utilization of Klenow and Taq DNA polymerase, the DSE heteroduplexes of wild type transcripts now contain the restriction enzyme site “Res”. Incubating the heteroduplexes with the restriction enzyme “Res” under conditions appropriate for enzyme activity results in the DSE heteroduplex of wild type being cleaved into two fragments. In contrast, the mutant heteroduplexes carrying a 5′ single nucleotide “A” overhang are unaffected by enzyme digestion due to lack of a “Res” site. Taking advantage of the 5′ single nucleotide “A” sticky ends of the mutant heteroduplexes, adapters with a complementary “T” sticky end can be ligated thereto while the “Res” digested heteroduplexes do not undergo ligation with the adapter. As described previously, any method known in the art may be used to detect and/or quantify the adapter-heteroduplexes such as PCR comprising use of a primer pair wherein one primer of the primer pair is unique to the adapter and the other primer is derived from the target gene of interest. The adapter may comprise a tag for detection and quantification of the adapter-heteroduplex. Though applicable for detection of any genetic mutation, the non-limiting method depicted in FIG. 4 may be well suited for detection of minimal residual disease such as B-cell and/or T-cell leukemia and lymphomas wherein monoclonally rearranged sequences are utilized as the mutant probes.

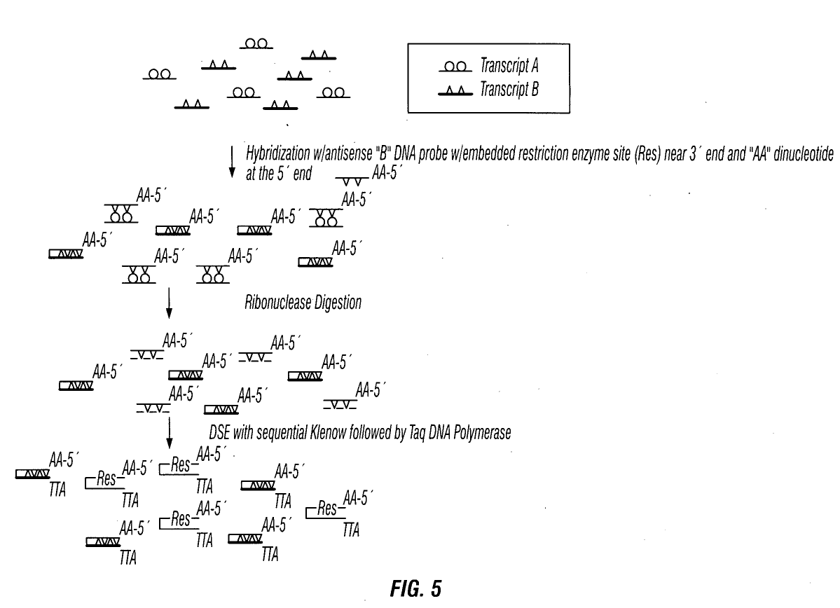

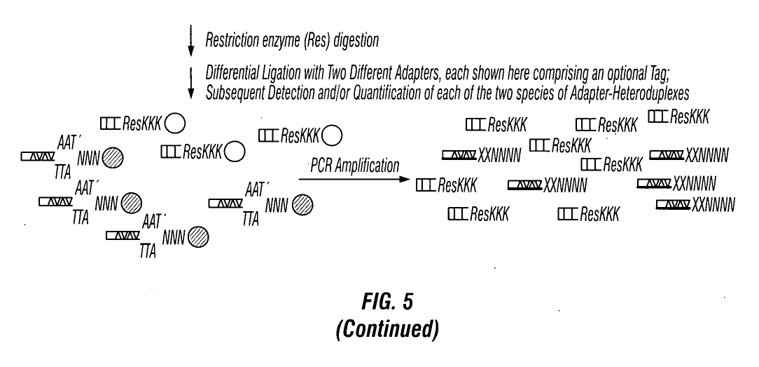

Referring now to FIG. 5, there is provided an overview of a non-limiting method for detecting a genetic mutation comprising use of two different types of adapters which allows for the subsequent detection and/or quantification of either or both the fully matched and partially mismatched RNA:DNA heteroduplexes. The general method is similar to those described in FIG. 4 until the step of ligation of adapter molecules at which point, two different species or types of adapters are included in the ligation reaction. One adapter has sticky ends complementary to those of the restriction enzyme digested heteroduplex. The second adapter has sticky ends different from those of the first adapter and which are complementary to the fully matched non-digested, and non-DSE heteroduplex molecules. Preferably the sequences of the two adapters are unique from one another, thus allowing each of the two different species of adapter-heteroduplexes to be detected and/or quantified. In the example depicted in FIG. 5, the first adapter, “ResKKK”, has “Res” sticky ends and thus ligates to the “Res” digested heteroduplex, while the second adapter, “TNNN”, has a single “T” overhang and thus ligates with the “A” sticky ended non DSE heteroduplexes (an “A/T” ligation). As with all the adapters used in the present disclosure, the adapters may comprise a tag to facilitate detection and/or quantification. If the adapters are tagged, the two species of adapters will preferably comprise tags different from each other thereby allowing for differential detection of each tagged-adapter-heteroduplex. The two different types of tags illustrated in FIG. 5 are depicted by filled circles and hatched circles. The method is easily adapted for use with more than 2 different species/types of adapters, each of which may comprise a unique tag. Though suitable for detection of any genetic mutation, the non-limiting method depicted in FIG. 5 may be well suited for detection and quantification of a polymorphism. For example in a mixed chimera post allogenic transplantation, perhaps especially useful for those individuals having a microsatellite polymorphism marker or a single nucleotide polymorphism marker available for use in discriminating between pre-transplant patterns and post transplant donor patterns.

In other non-limiting embodiments, the methods of the disclosure may be adapted for high throughput mutation screening such as, but not limited to, use of a microarray. Such an approach allows for mutation screening of a multitude of different genes simultaneously. Transcripts of interest include for example but not limited to, transcripts obtained by multiplex PCR target-specific amplification of genetic regions of interest, transcripts obtained by ubiquitous amplification of total cellular transcripts by with use of TMA or PCR, followed by transcription with RNA polymerases. The amplified transcripts may then be hybridized with a multitude of different single-stranded, antisense, wild type DNA probes. Generally the probes are deposited in microarray format on solid substrates suitable for high throughput for example, on slides or membranes, in multi-well plates or in tubes. The resulting heteroduplexes may then be treated with RNase. As an approach to reduce background noise, any of the blocking steps described previously may be used. Generally in the present embodiment, prior to performing the DSE step the heteroduplexes may be subjected to any one or more of the blocking steps of the disclosure, such as treatment with Klenow exo- and ddNTPs, described previously. The remaining steps of the method may be carried out as described previously for other method embodiments of the disclosure with at least one exception being that the present probes are in a microarray. In contrast to conventional micro-array technology, which requires use of hundreds of different mutant sequence combinations (i.e., probes) for each genetic region of interest, the microarray approach of the present disclosure requires only a single wild type probe per genetic region of interest in order to assay for any mutation therein.

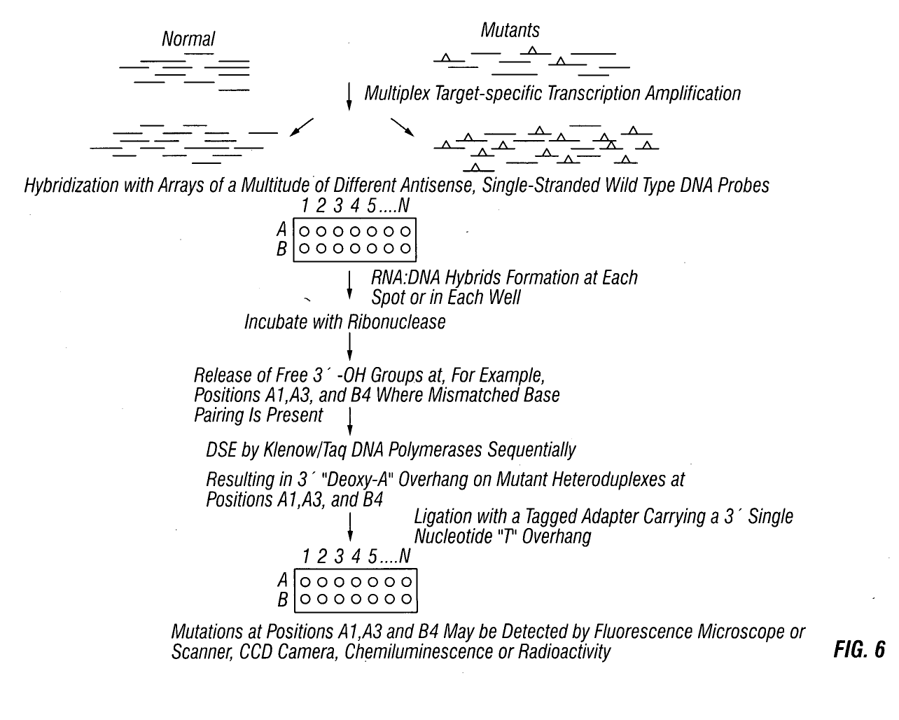

Referring now to FIG. 6, there is provided an overview of a non-limiting method for assaying for more than one genetic mutation at a time. Generally, at least one probe specific for each genetic region of interest is utilized. The probes may be immobilized onto any solid phase media known in the art, such as, but not limited to, a slide, a membrane, or a streptavidin-coated microwell plate. In FIG. 6, the probes are depicted as being arrayed on solid phase media such as a microwell plate in rows A and B comprising columns l through N. As indicated on FIG. 6, the RNA transcripts are derived by performing multiplex PCR using target-specific primers for simultaneous amplification of the genetic regions of interest. In order to from RNA:DNA heteroduplexes, aliquots of the DNA-free single stranded RNA transcripts are incubated with each sample of the arrayed DNA probe, shown in FIG. 6 as spots/wells on a plate. The remaining steps are similar to those described for other methods of the disclosure. By way of example, suppose in FIG. 6, that mismatched base paring occurs in at least one of the RNA:DNA heteroduplexes present at positions A1, A3, and B4. All samples are subject to RNase treatment followed by a DSE step with sequential utilization of Klenow and Taq DNA polymerases. The tagged adapter-DSE heteroduplexes of positive spots/wells A1, A3 and B4 may then be detected and quantitated using any methods suitable for the specific tag, for example, a fluorescence microscope, a scanner, a CCD camera, a chemiluminescence detector, or autoradiography.

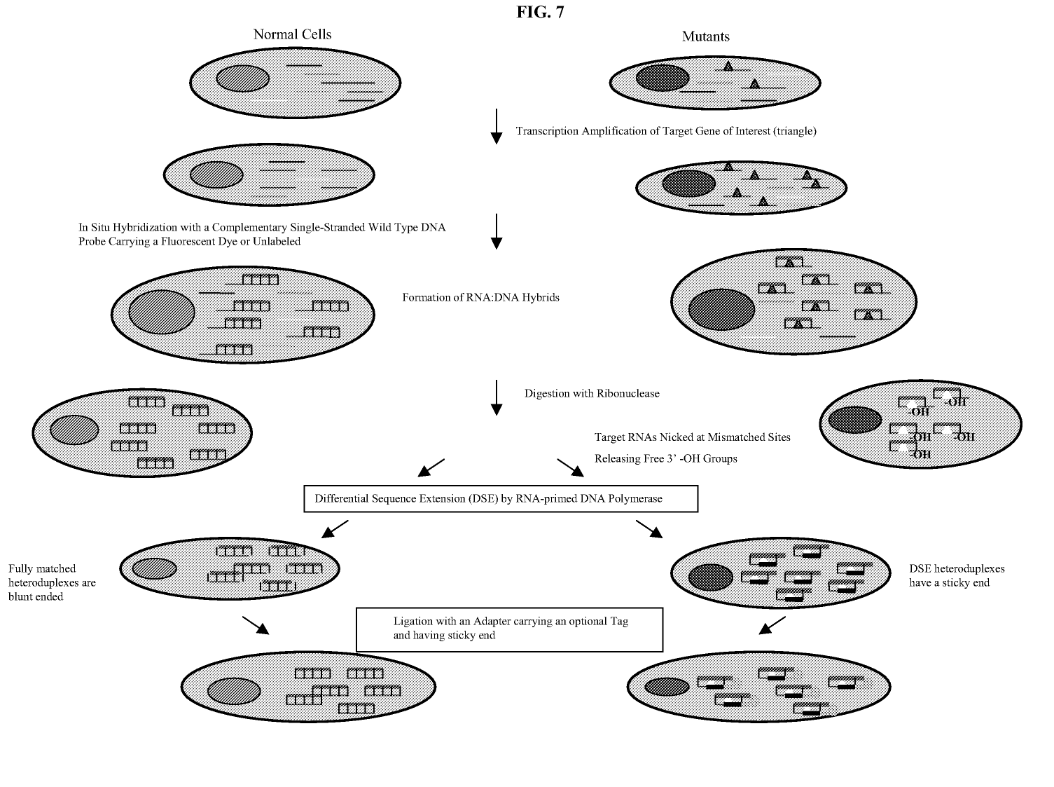

Referring now to FIG. 7, there is provided an overview of a non-limiting method for detecting genetic mutation wherein the method may be performed on samples such as tissue sections or cell preparations that are fixed onto slides. This non-limiting approach permits the detection of genetic mutations while preserving cell morphologies for microscopic examination. As can be seen in FIG. 7, the general method is similar to that described for FIG. 3 except the sample is fixed on a slide. As depicted in FIG. 7, after fixing the samples of interest on slides, target-specific transcripts (normal cells are depicted on the left side, mutants are on the right) are amplified by transcript-mediated amplification (TMA). The amplified transcripts may then be fixed by baking at 55° C. and alcohol treatment. After rehydration, the samples on the slides are subject to hybridization with an antisense, wild type DNA probe followed by ribonuclease digestion, and subsequent DSE. The DNA probe may be coupled with any suitable tag known in the art such as, but not limited to, a fluorescent dye. As explained in detail previously, the RNA-primed DNA polymerase use for DSE preferably leaves sticky ends on the DSE heteroduplexes. The fully matched heteroduplexes, which do not undergo DSE, remain blunt ended. An adapter having sticky ends complementary to those of the DSE heteroduplexes is then added to the sample under ligation conditions. The adapters undergo ligation to the DSE heteroduplex molecules but not to the fully matched heteroduplexes. The resulting DSE heteroduplex-adapter hybrids may be detected and/or quantified by any suitable method as described previously. If the adapter comprises a tag, the tag may be used for detecting and/or quantitating the adapter-heteroduplex molecules. By use of a fluorescent microscope or a regular light microscope, depending on the type of tag, attached to the adapter and/or the DNA probe, mutant cells can be identified and discriminated from normal cells while cell morphologies are also observed.

In non-limiting embodiments, part or all of the methods of the disclosure may be adapted for use with solid phase and/or immobilizable media such as but not limited to membranes, slides, plates, tubes, beads, chips, and combinations thereof. Immobilization of the nucleic acid may be by any suitable method known in the art, such as but not limited to, use of streptavidin, magnetism, microarrays/chips, and combinations thereof. Referring now to FIG. 8, there is provided an overview of a non-limiting method for mutation screening comprising use of solid phase media. The method may be applied for the analysis of a single target of interest, or for multiple targets of interest in multiple samples. Generally the method comprises ribonuclease treatment of RNA:DNA heteroduplexes of interest and subsequent DSE. Heteroduplexes are formed by hybridization of single stranded RNA transcripts of interest, together with antisense single stranded tagged DNA probes, which have been immobilized. For example, biotinylated tagged probes may be immobilized by use of a streptavidin coated surface/substrate.

Still referring to FIG. 8, the RNA transcripts may be obtained by first amplifying transcripts of interest in the samples of interest with a T7-M13-Target forward primer and a reverse primer carrying an embedded restriction enzyme site (“Res”). The T7-M13 forward primer, T7M13TF (+), carries T7 promoter sequence at its 5′ end, followed by an M13 sequence, and then the target specific sequence. The M13 sequence will be useful for any subsequent DNA sequencing. An oligonucleotide carrying a reverse complementary M13 sequence may be dually labeled with a fluorescent dye to serve as a ubiquitous probe for realtime PCR for the detection and quantification of mutant samples. Any suitable promoter sequences may be embedded in the primers used for PCR. The reverse primer, TR-Res(−) carries an embedded restriction enzyme site (“Res”) such as but not limited to BamHI, and the target specific sequence. Any suitable restriction enzyme site may be embedded in the primers for PCR. Multiplex PCR may be performed in order to analyze more than one target gene of interest. With current multiplex PCR technology, simultaneous amplification of about 6 to 8 target genes per reaction is possible. Thus, for amplification of, for example, 36 target genes of interest, as few as about 4 to 6 separate multiplex PCR reactions might be necessary for subsequent multiplex target-specific transcript amplification. An aliquot from each PCR, or a mixture of all the PCR products may then be subjected to transcript amplification by in vitro transcription using, in this example, T7 RNA polymerase.

For each target gene of interest, an antisense probe carrying a tag suitable for use with solid phase media, such as but not limited to a biotinylated tag, may be synthesized by PCR using a tagged TR-Res(−) primer as the primer and the T7M13TF/TR-Res amplicons of the previous step as templates. Mixtures of tagged probes may then be immobilized onto any solid phase media appropriate for the tag. For example, biotinylated probes may be immobilized onto wells of one or more streptavidin-coated microplate. The binding capacity of a streptavidin-coated microwell plate is typically more than 10 pmoles of biotinylated oligonucleotide probe. In a reaction volume of 25 microliters for each well of a plate, it is possible to accommodate 100 different probes at a final concentration of 4 nM for each probe.

Still referring to FIG. 8, probe hybridization to create RNA:DNA heteroduplexes, RNase digestion and the DSE step may be carried out as described previously with the exception being the probes and thus heteroduplexes formed therewith, are bound to solid phase media. In the example depicted in FIG. 8, the probes are wild type sequences. Following the DSE step, the mutant DNA:RNA heteroduplexes will have undergone DSE and will thus contain the restriction enzyme site, depicted in FIG. 8 as “Res”. Digestion with “Res” results in the release of the mutant DNA:RNA heteroduplexes from the solid media. The mutant DNA:RNA heteroduplexes may then be ligated to an adapter carrying a compatible “Res” sticky end and subjected to any one or more detection and/or quantitation technique. One approach is to carry out real-time PCR amplification using a forward T7 primer, a reverse primer derived from the adapter, and a dually fluorescence-labeled M13 probe in reverse sequence. Once the positive mutant amplicons are detected, they may be purified or cloned, and then sequenced by use of an M13 primer, thus allowing for possible identification of the genetic region and/or gene involved in the mutation.

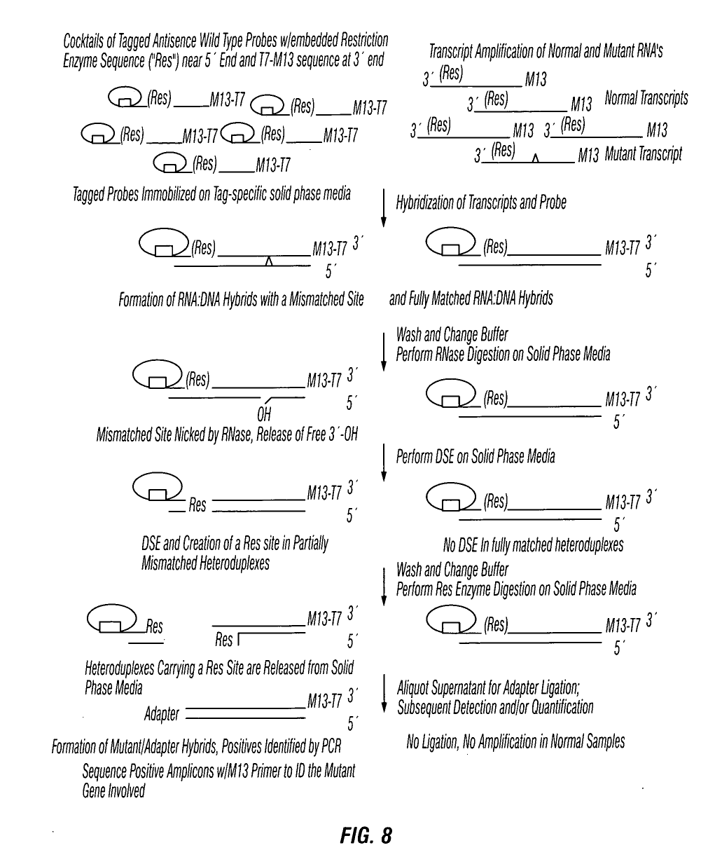

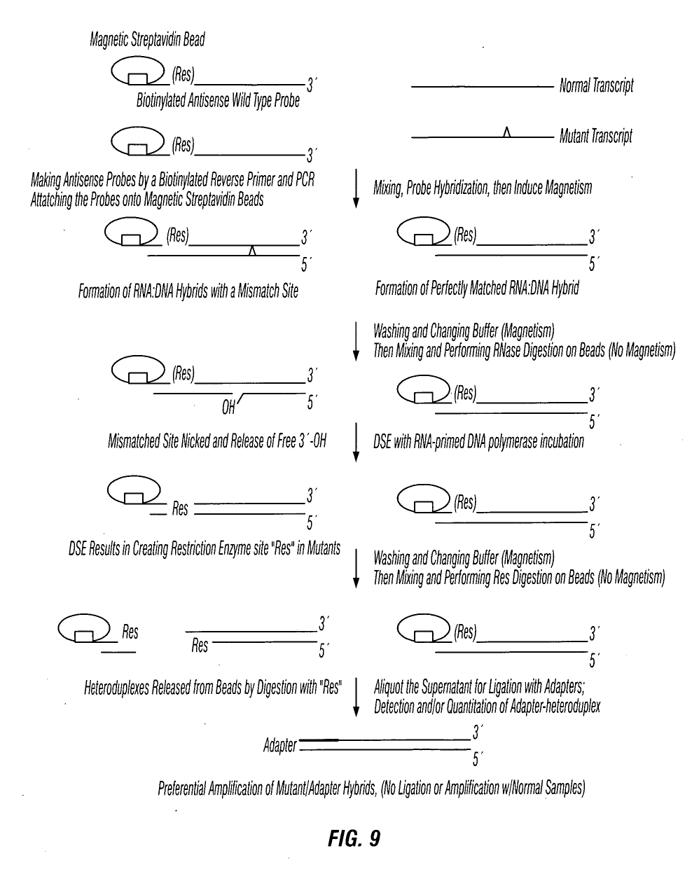

Referring now to FIG. 9, there is provided an overview of a non-limiting method for detecting mutation on solid phase media such as magnetic media. Generally, the single stranded antisense DNA probes used to create the RNA:DNA heteroduplexes of the disclosure are coupled with a magnetic tag, such as magnetic streptavidin beads, at their 5′ end and may also comprise a restriction enzyme site (“Res”) positioned 3′ of the tag useful in a subsequent step to release the probe from the tag. Generally the probes are produced by reverse transcription-mediated PCR (RT-PCR) wherein the reverse primer has a restriction enzyme site (“Res”) embedded near its 5′ end. The cDNA fragment resulting from RT-PCR may then be used as a template to synthesize a biotinylated antisense DNA probe by performing PCR using a biotinylated reverse primer. The reverse primer may be the same reverse primer used for RT-PCR with the exception being it has been biotinylated. As seen in FIG. 9, the resulting probe is biotinylated at its 5′ end and carries sequences for a “Res” site. The amplicons are then mixed with streptavidin magnetic beads in a reaction vessel, such as but not limited to a microcentrifuge tube following the manufacturer's instructions. The probe in FIG. 9 is depicted as wild type and will thus fully anneal to wild type target RNAs and be partially mismatched when hybridized to mutant RNAs.

As discussed previously, single stranded RNA transcripts used for creating the RNA:DNA heteroduplexes of the disclosure may be obtained by any applicable method known in the art. Generally, RT-PCR is performed to amplify the target genetic region of interest. Following in vitro transcription, the resulting transcripts of interest may be treated with DNase to produce DNA-free, transcripts of interest.

The DNA-free, single stranded RNA transcripts of interest are generally denatured prior to hybridization with antisense DNA probe. After denaturation, the transcripts of interest are subjected to hybridization conditions together with the magnetic bead-immobilized antisense wild-type probe. Following hybridization, any one or more blocking step of the disclosure may be carried out to reduce any background noise in the subsequent DSE step. Generally the blocking step is treatment with Klenow exo⁻ and ddNTP's to block any free 3′ OH groups, preventing nonspecific sequence extension in the subsequent DSE step. Optionally, additional blocking by TdT and ddNTP may be performed to ensure complete blockage of unwanted free 3′ OH groups before subjecting to ribonuclease digestion. The resulting DNA:RNA heteroduplexes are then treated with RNase followed by DSE with an RNA-primed DNA polymerase. Generally, the hybridization and enzymatic reactions are carried out in the absence of magnetism whereas washing steps are carried out in the presence of magnetism. This may be accomplished by physically adding and removing a magnet, or by turning on and off an electromagnetic field. Products for manipulation of magnetic particles are known and commercially available in the art. Washing in the presence of magnetic force allows removal of all molecules within the reaction vessel that are not bound to the magnetic beads. Following the sequence extension DSE step, the biotinylated DSE heteroduplexes may be digested with a restriction enzyme “Res” which releases the DSE heteroduplexes from the biotinylated tag. Following “res” digestion, in the presence of magnetism the supernatant containing the DSE heteroduplexes may be carefully aspirated from the reaction vessel microcentrifuge tube. As described previously, any detection and/or quantitation technique may then be carried out on the DSE heteroduplex molecules, such as ligation to an adapter having sticky ends complementary to the “Res” sticky ends for by any detection and/or quantitation methods, such as PCR amplification targeting the adapter-DSE heteroduplex molecules.

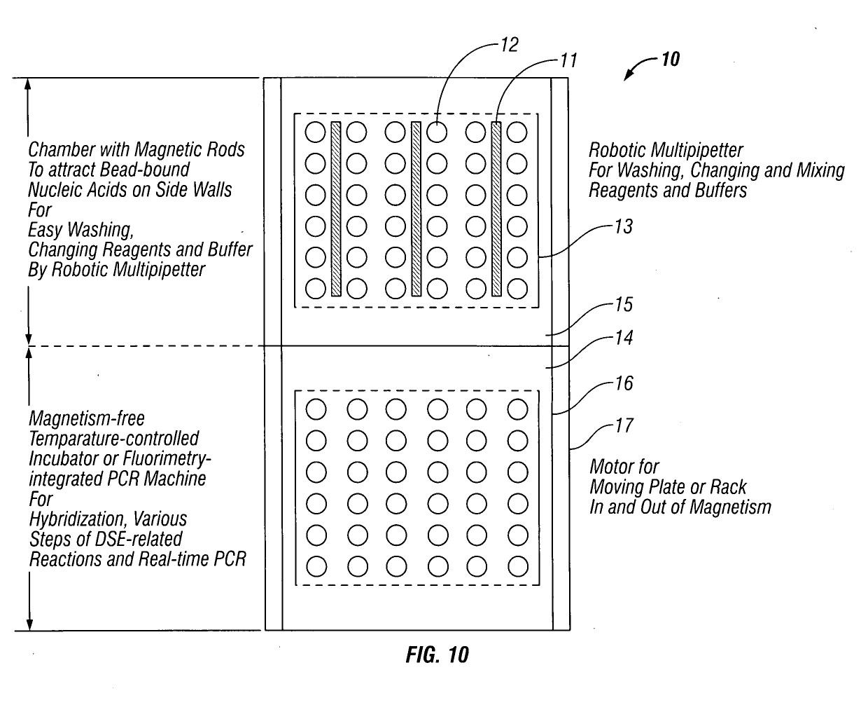

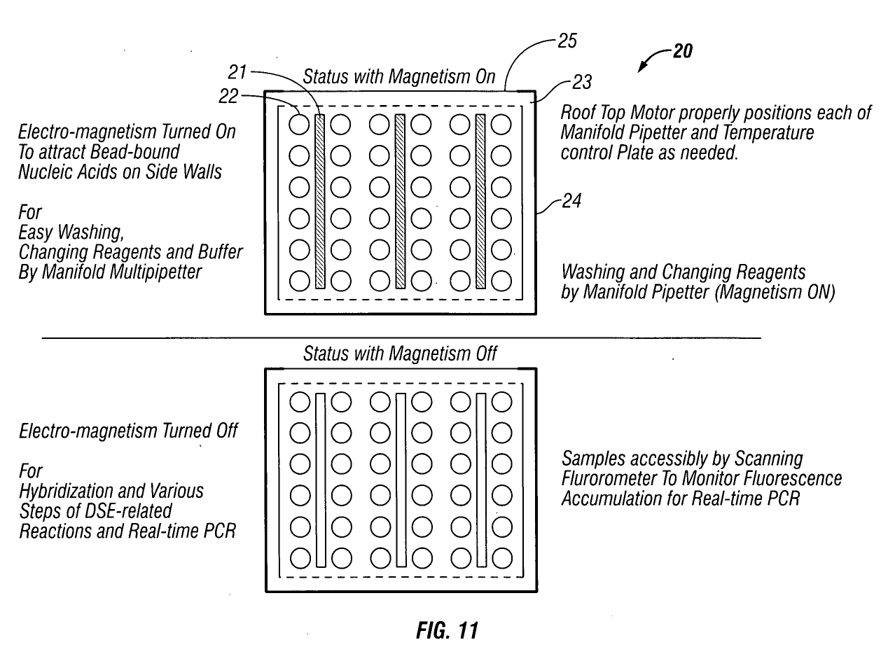

Referring now to FIGS. 10 and 11, aerial cross views of non-limiting apparatus suitable for carrying out methods of the disclosure comprising probes linked to an immobilizable tag, for example the method of FIG. 9, are depicted. Preferably the probes are linked to immobilizable tag having magnetic properties such as magnetic beads. Apparatus 10 and 20 respectively depicted in FIGS. 10 and 11 may be operated manually, automatically, or combinations thereof. Preferably apparatus 10 and 20 are automated. Apparatus 10 depicted in FIG. 10 comprises at least two reaction chambers, one of which comprises one or more magnetic members. Apparatus 20 depicted in FIG. 11 comprises one or more reaction chambers, wherein the one or more chambers comprise a magnetic element that can be turned on and off.

Focusing first on FIG. 10, apparatus 10 comprises chambers 13 and 14 enclosed by housing 17. Chambers 13 and 14 may be separated from one another by closing door 15. When closed, door 15 prohibits “cross-talk” between the chamber environments thereby producing two distinct chamber environments. The environments of chambers 13 and 14 are each temperature-controlled environments and may be set at different temperatures from one another. The chambers may each be set at any temperature necessary for processing of the samples. Samples 12 may be assayed in any suitable reaction vessel (not shown), including but not limited to, a well, a tube, a vial. If tubes or vials are used as the reaction vessels, generally they are placed in or on a rack, or platform, or the like, thus allowing large numbers of tubes or vials to be moved as a single unit, thereby saving time. If wells are used as the reaction vessels, generally multiwell plates are employed to allow movement of a multitude of wells/samples as a single unit. Chambers 13 and 14 may comprise any one or more reaction vessel holding device such as but not limited to a rack, shelf, platform, stand, and combinations thereof. Chambers 13 and 14 comprise at least one track 16, which may be used for directing movement of the reaction vessels and/or the reaction vessel holding device from one chamber to the other when door 15 is in an open position. The movement of samples between chambers 13 and 14 may be driven by a motor (not shown) via a swing arm. Chamber 13 of apparatus 10 comprises at least one magnetic member 11. Any number of magnetic members 11 may be utilized as long as contact between magnetic member 11 and each of samples 12 is optimized when samples are present in the chamber. In a similar regard, magnetic member 11 may have any shape as long as contact between magnetic member 11 and each reaction vessel of samples 12 is optimized when samples are present in the chamber. FIG. 10 depicts three magnetic members 11 each having a rod-like shape. Exposing samples 12 to magnetic members 11 in chamber 13 results in immobilization of the tagged probes within the samples. Chamber 13 is generally where the samples are washed and reaction reagents are added. The non-magnetic environment of chamber 14 is generally where enzymatic reactions and binding reactions are carried out. Apparatus 10 may further comprise an overhead robotic multipipetter (not shown) used for the addition and subtraction of solutions to each of the sample reaction vessels. Preferably the robotic multipipetter can access samples 12 in each of chambers 13 and 14.

Referring now to FIG. 11, apparatus 20 comprises chamber 23 enclosed by housing 24. Chamber 23 may be separated from the outside environment by closing door 25. Door 25 may slide or pivot between an open and a closed position and may do so vertically or horizontally. When closed, door 25 prohibits cross talk between the chamber environment and the room environment. The environment of chamber 23 is a temperature-controlled environment and may be set at any temperature necessary for processing of the samples. Chamber 23 may function as an incubator. Samples 22 may be assayed in any suitable reaction vessel (not shown), including but not limited to, a well, a tube, a vial. If tubes or vials are used as the reaction vessels, generally they are placed in or on a rack, or platform, or the like, thus allowing large numbers of tubes or vials to be moved as a single unit, thereby saving time. If wells are used as the reaction vessels, generally multiwell plates are employed to allow movement of a multitude of wells/samples as a single unit. Chamber 23 may comprise any one or more reaction vessel holding device such as but not limited to a rack, shelf, platform, stand, and combinations thereof. Chamber 23 may comprise any one or more reaction vessel holding device such as but not limited to a rack, shelf, platform, stand, and combinations thereof. Chamber 23 may comprise a means for directing automated movement of the reaction vessels and/or the reaction vessel holding device into and out of the chamber when door 25 is in an open position. The movement of samples may be driven by a motor (not shown) and an arm.

Chamber 23 of apparatus 20 comprises at least one electromagnetic member 21 wherein the magnetism of said member may be turned on and turned off. Any number of electromagnetic members 21 may be utilized as long as contact between electromagnetic member 21 and each of samples 12 is optimized when samples are present in the chamber. In a similar regard, electromagnetic member 21 may have any shape as long as contact between electromagnetic member 21 and each sample is optimized when samples are present in the chamber. FIG. 11 depicts three electromagnetic members 21 each having a rod-like shape. Electromagnetic members 21 may be turned on in order to induce magnetism and turned off in order to remove magnetism. The upper panel of FIG. 11 depicts electromagnetic members 21 as turned “on”, and the lower panel depicts them turned “off”. Exposure of samples comprising magnetically tagged probes to electromagnetic members 21 when said members are turned on results in immobilization of the tagged probes within the samples. Generally electromagnetic members 21 are turned on and the samples subjected to magnetism for the process steps during which the samples are being washed, and/or reaction reagents and/or buffers are being added to the reaction vessels. Electromagnetic members 21 are generally turned off when enzymatic reactions and binding reactions are carried out on samples 22. Apparatus 20 may also be used for non-magnetic solid phase media based DSE methods of the disclosure. If the solid phase media used to immobilize the probes within the samples are non-magnetic, for example streptavidin beads used to immobilize biotinylated probes, it is not necessary to utilize the electromagnetic rods.

Apparatus 20 may further comprise a robotic manifold multipipetter (not shown) positioned in a location that allows optimal access to the samples, such as located in the roof of the apparatus. The multipipetter is used to add and remove solutions/reagents to the sample reaction vessels, such as washes and buffer changes and addition of reaction reagents. Apparatus 10 and 20 may further comprise a temperature control element (not shown) used to control the temperature of the samples and positioned in a location that allows optimal access to the samples, such as located in the roof of the apparatus. In apparatus 10 and apparatus 20, both the pipetter and temperature control element may be driven by a motor that allows each of them to be moved out of the way when they are not in use. For example, when it is necessary to wash the samples and/or carry out buffer changes and/or add reagents, the motor will move the manifold pipetter into correct position above the sample reaction vessel. When a process step requires that the samples be subject to a temperature other than the temperature of the chamber environment, the motor will move the manifold pipetter out of the way, and position the temperature control element near, over or on top of the microwell plate in order to incubate samples at the desired temperature. The temperature control element may be any such element known in the art such as, but not limited to a such as a heating cover plate. Generally the temperature control element can subject the samples to any temperatures, preferably temperatures in the range of about 0 degrees C. to about 110 degrees C. Apparatus 10 and 20 may further comprise a fluorometer (not shown) allowing real-time PCR to be performed on the samples. A fluorometric scanner (not shown) will monitor fluorescence accumulation during PCR cycling. The fluorometer may be positioned in any location that allows accurate analysis of each of the samples. In one non-limiting embodiment, the fluorometer is located underneath the samples. Apparatus 10 and 20 may be designed to hold any number of samples and sample reaction vessels provided the apparatus design allows for uniform and consistent handling and reaction conditions for each sample therein, and allows accurate fluorometric analysis of each sample.

Other non-limiting embodiments of the present disclosure are directed to products for detecting genetic mutation. In one non-limiting embodiment there is provided a kit comprising reagents and a user's guide providing instruction for performing the methods of the disclosure. The methods of the disclosure may be provided in the user's guide comprising hardcopy printed literature, computer readable media, and combinations thereof. Generally the kit provides the user's guide in a hardcopy printed form and a computer readable form, for example, a CD-ROM or DVD. The kits may be custom-made for analysis of any or more genetic regions of interest, the kits may comprise reagents and materials for broad range mutational screening applications, or the kits may comprise reagents and materials to detect any of a number of mutations commonly associated with and/or observed in any one or more condition or disease, such as a cancer. Generally the reagents and materials of the kit include the probes necessary to screen genetic regions of interest. The probes may be synthesized as described previously in the disclosure. The probes may be coupled with any tag as described in detail previously. The probes may be a mixture of different probes provided in solution, or a multitude of probes attached to a solid phase media as was described elsewhere in the present disclosure. In one non-limiting embodiment, the probes provided in the kit are coupled with an immobilizable tag and are provided in the kit suspended in an appropriate buffer solution. In another non-limiting embodiment, the probes provided in the kit are provided in an array on a solid phase media, such as deposited in an array on one or more microwell plates, or as a DNA microarray/chip. Any methods known in the art for producing DNA arrays may be used to create the DNA microarrays of the present disclosure. Any RNA samples may be assayed with the kits of the disclosure provided the RNA transcripts are prepared according the instructions of the user's guide and as detailed previously in the disclosure in the discussion of transcript preparation. Generally the kits may be selected from at least one of the following groups: 1) a kit specific for screening for mutation in oncogenes and comprising a mixture of probes specific for oncogenes such as but not limited to K-ras, N-ras, H-ras, Src, Bcr-Abl, Myc; 2) a kit specific for screening for mutation in tumor suppressor genes and comprising a mixture of probes specific for known tumor suppressor genes such as, but not limited to TP53, P16, Rb1, NF1; 3) a kit specific for screening for mutation in mismatch repair genes and comprising a mixture of probes specific for mismatch repair genes, such as but not limited to MLH1, MSH2, MDM2, MYH, ATM, BRCA1; 4) a kit specific for screening for mutation in tyrosine kinase genes also called “Tyrosine Kinome” and comprising a mixture of probes specific for tyrosine kinase genes such as but not limited to NTRK2, NTRK3, FES, KDR, EPHA3, MLK4, GUCY2F, c-KIT, FLT3, JAK2; 5) a kit specific for screening for mutation in growth factor receptor genes and comprising a mixture of probes specific for growth factor receptor genes, such as but not limited to EGFR, PDGFR□, PDGFR□, VEGFR1, VEGFR2, TGF□1, TGF□2; 6) a kit specific for screening for mutation in mitochondrial DNA, either within D-loop or non-D-loop (coding) regions and comprising a mixture of probes specific to those regions; 7) a kit specific for screening for mutation in SNP markers and comprising a mixture of probes specific for SNP markers; 8) a kit specific for screening for mutation in microsatellite polymorphism markers and comprising probes specific for microsatellite polymorphism markers; and 9) a kit specific for screening for mutation in immunoglobulin superfamily genes and comprising a mixture of probes specific for immunoglobulin superfamily genes such as but not limited to immunoglobulin heavy chain gene and kappa light chain gene, T-cell receptor [symbol] genes.

In addition to user's guide and probes, the kits of the disclosure may further comprise any one or more of at least a pair of primers specific for each of the genetic regions of interest targeted by said kit, a reverse transcriptase and buffer, an RNA polymerase and polymerase buffer, a DNase and DNase buffer, and combinations thereof, for preparation and amplification of the RNA transcripts to be used in hybridizations with the probes of the kit to create RNA:DNA heteroduplexes according to the instructions in the user's guide. Any one or more of the primers described in the present disclosure may included in the kits. The kits of the disclosure may be designed to further comprise blocking reagents useful for blocking background after the hybridization of RNA:DNA heteroduplexes. The blocking reagents may include but are not limited to, any one or more of the following: Klenow enzyme and Klenow buffer and ddNTPS, Klenow exo- and Klenow exo- buffer and ddNTPs, TdT and TdT buffer and ddNTPS, purification columns to purify products following the blocking step, and any combinations thereof. The kits of the disclosure may be designed to further comprise linking adapters useful for DSE-mediated ligation of said adapters to the DSE-heteroduplexes following the DSE step. Any one or more of the linking adapters described in the present disclosure may be included in the present kits. The kits of the disclosure may be designed to further comprise but are not limited to, any of the following: RNase enzyme and buffer for the RNase treatment step, Klenow and buffer for the DSE step, Klenow exo- and buffer for the DSE step, Taq and buffer for the DSE step, and any combinations thereof.

In other non-limiting embodiments, any one or more steps of the methods of the disclosure may be adapted for automation and/or batch production. Any applicable automated apparatus may be used to perform any one or more process step of the methods of disclosure by use of any applicable technology known in the art, for example, robotic arms to move elements, robotic pipettes to add, mix, and/or remove reagents, reaction chambers enabling control of environmental parameters such as temperature and magnetism, movable trays, racks and/or platforms for movement of samples and reaction vessels. Automation of the methods of the disclosure permits high throughput mutation screening on multitudes of different samples simultaneously.

Unless stated otherwise, the practice of the present disclosure makes use of molecular biology, microbiology and recombinant DNA techniques. All general and support techniques utilized and applicable herein are explained fully in the literature. The reagents and machinery for PCR, probe hybridization, nuclease digestions including but not limited to ribonucleases, and S1 nucleases, restriction enzyme digestions, DNA and RNA polymerase reactions, ligations, amplification of transcripts, purification methods, detections methods, quantification methods, and all other molecular/recombinant biology techniques are known by one of skill in the art and are suitable for use herein. Simple adjustments made to known reaction variables, such as reaction temperatures and times, and the design of oligonucleotide primers for PCR, and the design of adapters whether tagged or untagged, should not be misconstrued as undue experimentation but rather understood to be within the skill of one in the art.

With respect to PCR reactions in particular, these may be carried out with any equipment or apparatus suitable for thermo-cycling. Generally, the PCR reactions comprise between 1 and 60 cycles, preferably between 5 and 55 cycles and more preferably between 10 and 50 cycles. The first step of the cycle is carried out at a temperature generally between 90 and 98 degrees C., for a time period between 1 and 60 seconds. The second step of the cycle is carried out at a temperature generally between 50 and 65 degrees C., for a time period between 1 and 60 seconds. The third step of the cycle is carried out at a temperature generally between 65 and 75 degrees C., for a time period generally between 10 and 120 seconds. As known in the art, as an approach to optimize the reaction, the cycles may be preceded by an initial heat activation of the DNA polymerase used therein, for example when a hot start DNA polymerase is used, such as but not limited to Taq DNA polymerase GOLD. In one non-limiting embodiment, PCR is carried out with an ABI 7900HT Sequence Detection System (Applied Biosystems, Foster City, Calif.) and comprises 45 cycles of the following steps: 95 degrees C. for 30 seconds, 60 degrees C. for 30 seconds, and 72 degrees C. for 45 seconds, and includes an initial heat activation of Taq DNA polymerase GOLD for 10 minutes.

The present disclosure is to be taken as illustrative rather than as limiting the scope or nature of the claims below. Numerous modifications and variations will become apparent to those skilled in the art after studying the disclosure, including use of equivalent functional and/or structural substitutes for elements described herein, use of equivalent functional substituents for substituents described herein, and/or use of equivalent functional reactions for reactions described herein. Any insubstantial variations are to be considered within the scope of the claims below. All literature including patent applications, provisional applications, and journal articles cited herein is hereby incorporated by reference.

### EXAMPLES

### Example 1

### Detection of a Point Mutation in the K-Ras Oncogene by DSE-Mediated Ligation and Amplification

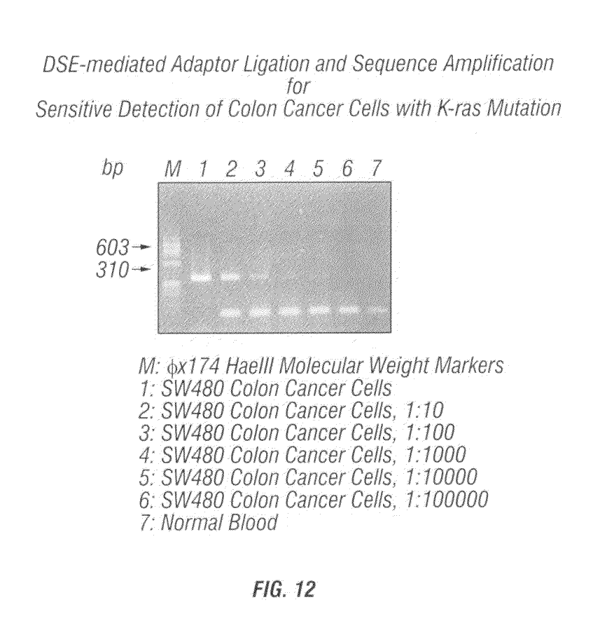

The present example illustrates one non-limiting application of the DSE-applied assay for detecting point mutations and utilizes SW480 colon cancer cells and normal blood samples and results are provided in FIG. 12. SW480 colon cancer cells were selected because they are well known to carry a homozygous point mutation at codon 12 of the K-ras oncogene. Five serial dilutions (1:10, 1:100, 1:1000, 1:10000, 1:100000) (lanes 2-6, respectively, of FIG. 12) of the SW480 cells were utilized in the present example to investigate the sensitivity of the DSE method.

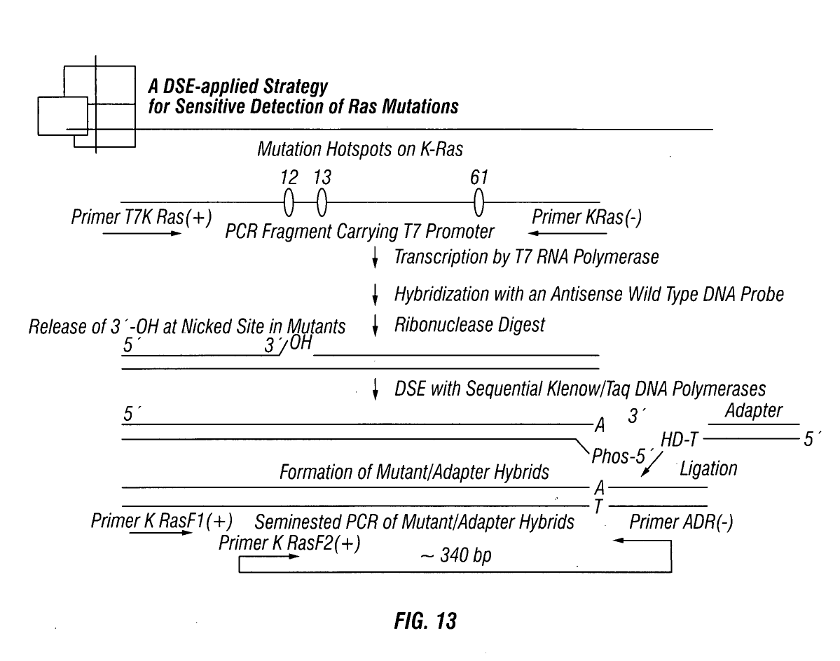

Following RT-PCR using a forward T7K-ras(+) primer, 5′-TAATACGACTCACTATAGGGCCTGCTGAAAATGACTGAA-3′, and a reverse K-ras(−) primer, 5′-TACTAGGACCATAGGTACAT-3′, the resulting T7K-ras cDNA amplicons were subjected to transcription with T7 RNA polymerase. The resulting K-ras transcripts were then treated with DNase. An antisense K-ras probe was synthesized by PCR using a 5′-phosphorylated reverse Kras(−) primer 5′-[Phos]-TACTAGGACCATAGGTACAT-3′, and the T7K-ras cDNA of a normal sample as a template. The resulting products were digested with HhaI and HpaII, blocked with Klenow exo- and ddNTP's, and ligation with a blocking adapter. The blocking adapter was formed by annealing of two complementary oligonucleotides: B-Adp(+), 5′-[Phos]-CCTGCAGGAGACGGTGA-[Phos]-3′, and B-Adp(−), 5′-TCACCGTCTCCTGCAGGT-3′. The resulting double-stranded adapter carried a 3′ single nucleotide “T” overhang. The sense strand of the blocking adapter was dually phosphorylated. The 5′ phosphorylation was to provide a phosphate group for ligation with any PCR products carrying a 3′ single nucleotide “A’ tail. The 3′ phosphorylation was to prevent the ligation products from sequence extension by subsequence DSE reaction. Following purification by a Quiaquick column, the 5′-phosphorylated antisense K-ras probe was subjected to hybridization with the T7 transcribed K-ras RNA's of the tested samples. Blockage with Klenow exo- and ddNTP's was then carried, followed by RNase ONE digestion. After purification with a Quiaquick column, DSE was performed by sequential utilization of Klenow enzyme and taq DNA polymerase. As a result, the DSE products of K-ras mutants carried a 3′ single nucleotide “A” overhang owing to nicking at the mismatched site, new DNA strand extension primed from the nicked site, and adding of a single nucleotide “A” protruding end at the 3′ end of the new DNA strand. In contrast, samples with normal K-ras were not cleaved, thereby no DSE products were formed and remained blunt ended. The DSE products were then ligated with an adapter carrying a 3′ single nucleotide “T” overhang. The adapter was formed by annealing of two complementary oligonucleotides: Adp(+), 5′-GAGCGAGCAGCAGCTG-3′, and Adp(−), 5′-CAGCTGCTGCTCGCTCT-3′. The ligation products were treated with S1 nuclease, purified by a Quiaquick column, and then subjected to semi-nested PCR amplification using primers K-rasF1(+), 5′-TGGTAGTTGGAGCTTGTGG-3′, and Adp(−) for the first round of PCR, and primers K-rasF2(+), 5′-TAGGCAAGAGTGCCTTGAC-3′, and Adp(−) for the second round of PCR. The expected amplicons of the K-ras mutants/adapter hybrids were ˜340 bp in size. As shown in FIG. 12, positives were detected in the SW480 cells (lane 1) and the serially diluted samples (lanes 2-6), but not in the normal blood sample (lane 7). Remarkably, a faint band of expected size was detected in the 1:100,000 dilution SW480 sample (see lane 6 of FIG. 12) reflective of the sensitivity of the present method. FIG. 13 provides a schematic of the present example.

### Example 2

### Detection of Tyrosine Kinase (TK) Mutations by the DSE-Applied Assay in Philadelphia Chromosome (Ph) Positive CML Patients on TK Inhibitor Therapy

### Protocol #1

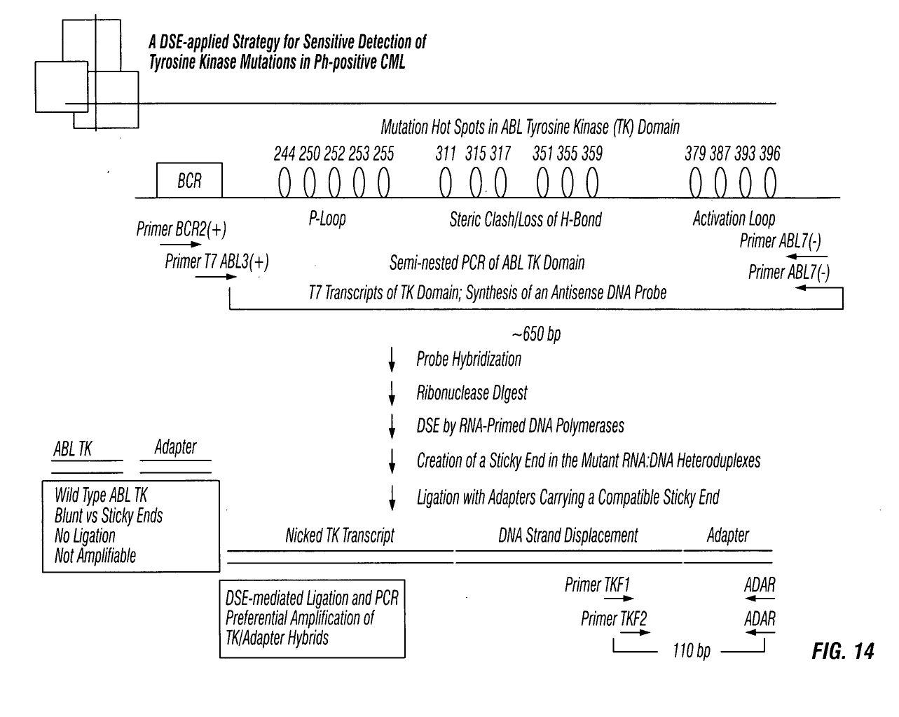

The present non-limiting example illustrates a use of the DSE assay for detecting TK mutations in Philadelphia chromosome (Ph) positive CML patients. A known example of mutation-conferred resistance is mutation in the BCR/abl tyrosine kinase in Ph-positive CML patients after treatment with TK inhibitors such as Imatinib. It is known in the art that TK mutation hotspots are spread throughout the TK domain of the c-abl gene. As an approach to ensure the analysis covered all potential mutation hotspots, an approximately 650 base pair (bp) cDNA fragment that spans the TK domain of the c-abl gene was analyzed. The mutation hot spots examined and primers used in this DSE-applied assay are schematically illustrated in FIG. 14.

Total cellular RNAs are extracted from the bone marrow samples obtained from Ph-positive CML patients receiving treatment with a TK inhibitor, such as Imatinib. Following reverse transcription with random hexamers, the resulting cDNAs are subject to semi-nested PCR amplification. In the first round of PCR, a forward primer derived from the second exon of the BCR region, BCR 2(+)5′-CATTCCGCTGACCATCAAT-3′, and a reverse primer derived from the seventh exon of the c-abl gene, ABL 7(−)5′-ACGTCGGACTTGATGGAGAA-3′, are used to amplify the cDNAs of BCR/abl fusion transcripts of the Ph chromosome. In the second round of PCR, a forward primer derived from the third exon of the c-abl gene with T7 promoter sequences attached to its 5′ end, T7ABL3(+) 5′-TAATACGACTCACTATAGGGATCATTCAACGGTGGCCGAC-3′, and the ABL 7(−) reverse primer are used to amplify an approximately 650 base pair (bp) cDNA fragment that spans the TK domain of the c-abl gene. Using PCR with 5′ phosphorylated ABL7(−) as a primer and the ABL TK amplicon from a normal control sample as a template, an antisense single-stranded wild type TK probe is synthesized. As described above, the resulting probe is digested with frequent cutting restriction enzymes, HhaI and HpaII, blocked with Klenow exo- and ddNTP, and then ligated with an blocking adapter prior to hybridization with the TK transcripts of the tested samples. The blocking adapter is formed by annealing two complementary oligonucleotides, BLK-Adp(+) 5′-{Phos]GTCCTCATGTACTGGTC[Phos]-3′ and BLK-Adp(−) 5′-GACCAGTACATGAGGACT-3′.

Taking advantage of the T7 promoter sequences in the resulting ABL TK cDNA fragments, TK transcripts are synthesized with T7 RNA polymerase. The amplified transcripts are then hybridized with the above-mentioned antisense, wild type DNA probe complementary to the TK transcripts in a hybridization buffer containing 10 mM Tris pH 7.5, 1.25 M NaCl, and 5 mM EDTA at 70° C. for 1 hour. As a result, fully matched, normal RNA:DNA heteroduplexes are formed as opposed to partially mismatched heteroduplexes in TK mutants.

Prior to ribonuclease digestion, blockage with Klenow exo⁻, TdT and ddNTP's is performed at 37° C. for 4 hours. The ddNTP blockage after the formation of RNA:DNA heteroduplexes prevents partially transcribed RNAs and any free 3′-OH from sequence extension in later steps. After purification with a Qiaquick spin column, the resulting RNA:DNA heteroduplexes are digested with 10 units of RNase ONE in an optimized buffer (Promega, Madison, Wis.). After ribonuclease digestion, the partially mismatched heteroduplexes are nicked at the mismatched site on the RNA strands, resulting in the release of free 3′-OH groups that, in turn, serve as primers for sequence extension in a reaction cocktail containing 5 units of Klenow enzyme, 5 units of Taq DNA polymerase, 50 mM NaCl, 5 mM MgCl₂, 250 M of deoxyribonucleotide mix (dNTP's), and 5 mM DTT at 37 C for 30 minutes, and then at 70 C for another 30 minutes. In contrast, fully matched, normal heteroduplexes are completely protected from ribonuclease hydrolysis, thereby preventing further sequence extension. This contrasted difference has therefore been referred to as Differential Sequence Extension (DSE). DSE through sequential utilization of Klenow/Taq DNA polymerases results in a 3′ single nucleotide “deoxy-A” overhang because Taq polymerase has terminal deoxynucleotide transferase (TdT) activity that preferentially incorporates a “deoxy-A” at the 3′ end of its sequence extension without the need of a DNA template. Taking advantage of the presence of a 3′ single nucleotide “A” overhang the DSE products are ligated with the above mentioned adapter that carries a complementary 3′ single nucleotide “T” overhang, resulting in the formation of mutant/adapter hybrids. Since the sequences of the TK domain and the adapter are known (and available for example from GenBank), Semi-nested PCR is performed by using primers derived from the TK domain and the adapter to preferentially amplify the mutant/adapter hybrids, thereby permitting specific and sensitive detection of small numbers of TK mutants in CML patients receiving TK inhibitor therapy.

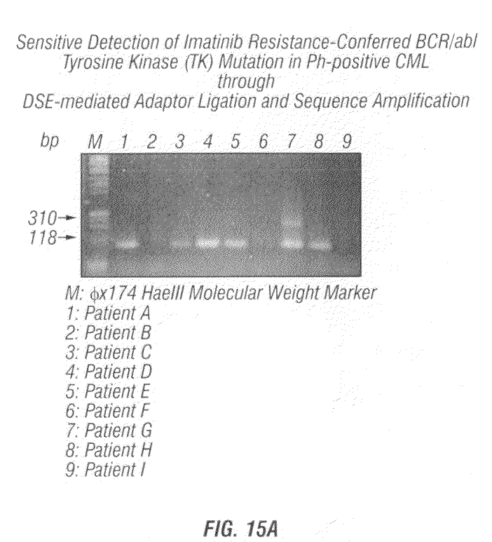

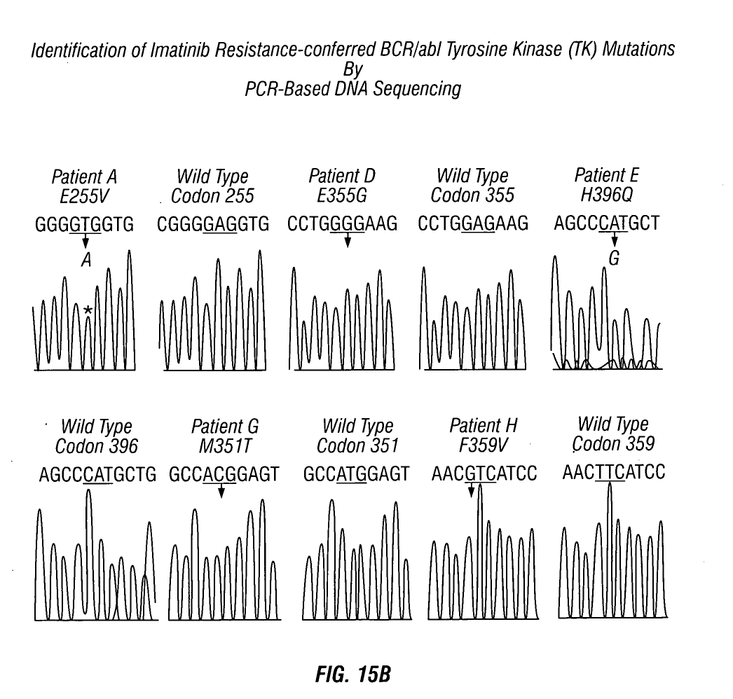

In the first round of PCR, primer TKF1(5′-GAGAACCACTTGGTGAAGGT-3′) and primer Adp(−), were used. In the second round of PCR, primer TKF2 (5′-TGAGCAGGTTGATGACAGG-3′) and primer Adp(−) were used. The expected PCR products of the mutants/adapter hybrids were ˜110 bp in size. Shown in FIG. 15 are nine representative Ph-positive CML patients in various clinical statuses following Imatinib therapy. Of those nine, five (Patients A, D, E, G and H) (FIG. 15A, lanes 1, 4, 5, 7 and 8, respectively) had Imatinib resistance with Ph chromosome >35% and TK mutations confirmed by PCR-based DNA sequencing; two (Patients C and F) (FIG. 15A, lanes 3 and 6) were in cytogenetic remission with detectable residual BCR/abl transcripts by qRT-PCR; and the remaining two (Patients B and I) (FIG. 15A, lanes 2 and 9) were in cytogenetic remission without detectable BCR/abl fusion transcripts.

As can be seen in FIG. 15A, strong positive signals were detected in Patients A, D, E, G, and H (see lanes 1, 4, 5, 7 and 8) who were clinically resistant to Imatinib. The TK mutations in these five patients were also detected by DNA sequencing, results shown in FIG. 15B. While Patients B and I were negative, Patient C had a weak positive band and Patient F had a questionable positive that was later demonstrated by DSE-applied real-time PCR (see Example 3).

In total, 27 samples have thus far been screened for mutation by the method (protocol #1) described above. Table 1 summarizes the DSE results and DNA sequencing results. Of the 27 samples, nine were identified as positive for TK mutation by the present inventive DSE assay, and the mutations were confirmed by DNA sequencing. Three of the samples were identified as positive for TK mutation in the DSE assay, but negative for mutation according to DNA sequencing. Fifteen of the samples were identified as negative for TK mutation in the DSE assay, and were confirmed as negatives by DNA sequencing.

TABLE 1
TK mutations results: Comparison of DSE assay vs DNA Sequencing

| | Sequencing (+) | Sequencing (−) | Total |
|---|---|---|---|
| DSE(+) | 9 | 3 | 12 |
| DSE(−) | 0 | 15 | 15 |
| Total | 9 | 18 | 27 |

Degrees of freedom: 1
Chi-square = 16.875
p is less than or equal to 0.001
Distribution is significant

### Example 3

### Sensitive and Quantitative Detection of TK Mutants by DSE-Applied Real-Time PCR for the Mutants/Adapter Hybrids in Ph-Positive CML Patients on TK Inhibitor Therapy

In light of the variations in the intensities of the positive signals detected by the DSE-applied assay, a quantitative real-time PCR assay was developed in order to examine the patients. To establish a standard serial dilution plot for quantification, a hybrid construct (110 bp in size) in which the 5′ ninety nucleotides were derived from the 3′ region of the abl TK domain and the 3′ twenty nucleotides were derived from the adapter sequences was created. The following quantities of the hybrid constructs were used to make a standard curve plot: 10, 10², 10³, 10⁴, 10⁵, 10⁶, 10⁷, and 10⁸ copies. Real-time PCR was performed on the first PCR products described in Example 2, Protocol 1, in an ABI 7900HT sequence detector using a forward primer, TKF2, the reverse ADAR primer described in Example 2, Protocol 1, and a dually fluorescence-labeled probe (designated as ABLTKR) that carries a 6-FAM dye at its 5′ end and a TAMRA dye at its 3′ end.

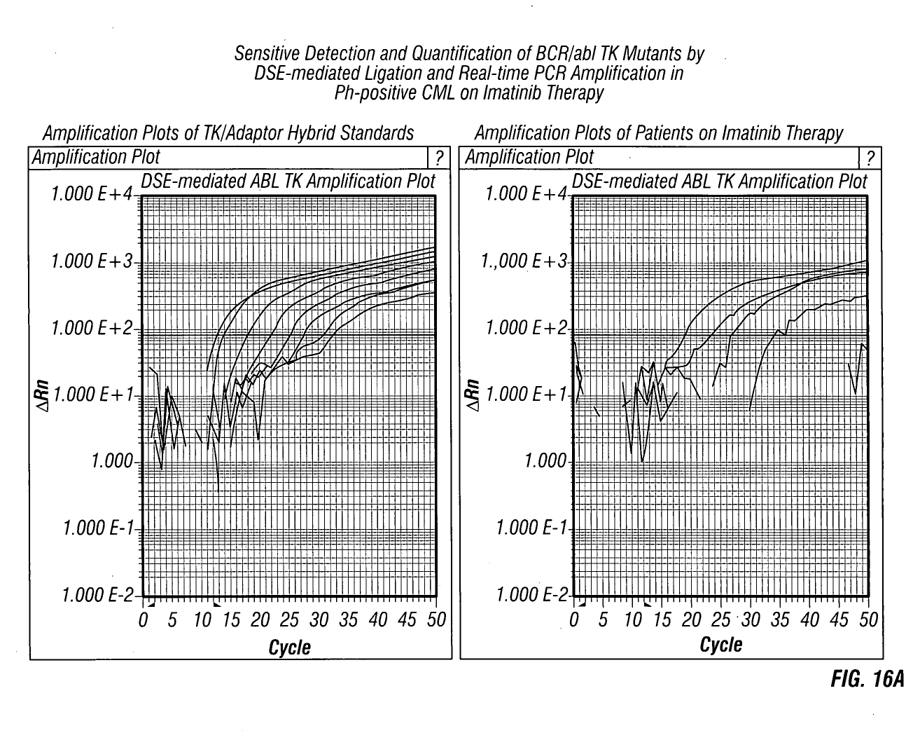

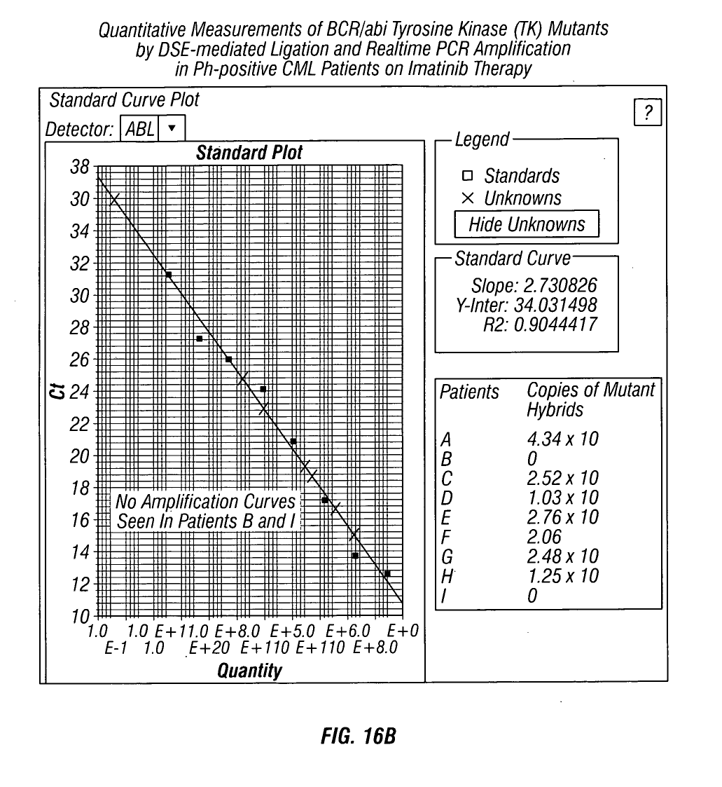

Shown in the left panel of FIG. 16A are the amplification plots of serial dilutions of the TK/adapter hybrid standards and shown in the right panel are the amplification plots of five representative patients (A, B, F, G, and H). (Patients A, B, F, G, and H are lanes 1, 2, 6, 7, and 8 of FIG. 15A). While Patient B was negative, positive amplification curves in various intensities were detected in Patients A, F, G and H. Intriguingly, although the presence of a TK mutation in Patient F was questionable by gel electrophoresis following DSE assay (see lane 6 FIG. 15A), a weak amplification curve was clearly and convincingly detected by real-time PCR (FIG. 16A), indicating the presence of a very low quantity of TK mutants.

Except for patients B and I where there were no amplification curves detected, the quantitative measurements of the TK mutants in the remaining seven patients were shown with the standard curve plot in FIG. 16B. The quantities of the TK mutant/adapter hybrids in Patients A, C, D, E, F, G and H were estimated as 4.34×10⁵, 2.52×10³, 1.03×10⁷, 2.76×10⁵, 2.06, 2.48×10⁶, 1.25×10⁴ copies respectively.

DSE-applied realtime PCR quantification of BCR/abl TK mutants may be performed by comparison against the standard curve plots established by a set of serially diluted positive samples (1:10, 1:10², 1:10³, 1:10⁴, 1:10⁵1:10⁶) prepared from a patient who carried 100% Ph chromosome and an BCR/abl TK mutation documented by DNA sequencing. Twenty-six bone marrow samples were analyzed by protocol 1 using this calculation method. Of the 26 samples, five harbored a TK mutation as determined by DNA sequencing. The quantities of TK mutants were estimated as 3.718, 3.314, 2.36, 0.376 and 0.17, respectively. The mutation was characterized as N311 S, M351T, H396Q, F317L, and F317L, respectively. There were seventeen samples showing negative results as determined by either the DSE-applied assay or by DNA sequencing. The remaining four samples showed weak positives by the DSE-applied assay while DNA sequencing was negative. The quantities of the TK mutants were estimated as 1.22×10⁻¹, 6.7×10⁻², 2.5×10⁻², and 2.5×10⁻³, respectively.

One additional DSE-applied laboratory protocol has been developed to detect and quantify BCR/abl TK mutants in Ph-positive CML patients on Imatinib therapy. It involves the creation of the sticky ends of a restriction enzyme site (“Res”) for subsequent adapter ligation. Although these two protocols share the same basic principles, there are also variations between them such as the use of reverse primers for PCR and synthesis of antisense wild-type DNA probes, and blocking non-specific ligation and background noise. These protocols are easily adapted to any gene of interest or genetic region of interest simply by tailoring the reactions agents such as, but not limited to, primers, probes, and adapters to fit the gene or genetic region of interest.

### Protocol #2

PCR Amplification and T7 Transcription of the BCR/abl TK Domain—Following reverse transcription on the total cellular RNAs prepared from patients' samples, the resulting cDNA's are subject to semi-nested PCR amplification of the BCR/abl TK domain. In the first round of PCR, a forward BCR2(+) primer, 5′-CATTCCGCTGACCATCAAT-3′, and a reverse ABL7-Bam(−) primer, 5′-TAGGGGAACTTGGATCCAGC-3′, are used to amplify the cDNA's of BCR/abl fusion transcripts of the Ph chromosome. The sequences of BCR2(+) were derived from the second exon of the BCR region. And the sequences of ABL7-Bam(−) were derived from the seventh exon of the c-abl gene plus an embedded BamHI site. In the second round of PCR, forward primer T7ABL3(+) and reverse primer ABL7-Bam(−) are used to amplify a ˜650 base pair (bp) cDNA fragment that spans the TK domain of the c-abl gene. The sequences of T7ABL3(+), 5′-TAATACGACTCACTATAGGGATCATTCAACGGTGGCCGAC-3′, are derived from the third exon of the c-abl gene with T7 promoter sequences attached to its 5′ end. After PCR, the amplicons are purified with a QIAquick PCR purification kit (QIAGEN, Valencia, Calif.), then subject to RNA transcription with T7 RNA polymerase in a reaction cocktail containing 750 M of ribonucleotide mix (rATP, rCTP, rGTP, and rUTP), 5 mM of dithiothreitol (DTT), 40 units of T7 RNA polymerase, and 20 units of RNasin, followed by DNase digestion to attain DNA-free, ABL TK RNA transcripts.

Preparation of an Antisense Single-Stranded Wide-Type DNA Probe for ABL TK—Total cellular RNAs of a normal individual are reverse transcribed, then seminested PCR is performed to amplify a wild-type ABL TK cDNA fragment as described above. The cDNA is then used as a template to synthesize an antisense, single-stranded DNA probe by PCR and the reverse primer ABL7-Bam(−). To eliminate unwanted double-stranded amplicons and hidden BamHI sequences, a cocktail of restriction enzymes containing either “HpaII and MboI”, or “HpaII, HhaI, and BamHI”, is added to the PCR products after PCR. The frequent-cutting restriction enzymes, HpaII, MboI, and HhaI, digest double-stranded DNA amplicons into smaller pieces. MboI and BamHI digestions expose BamHI sticky ends, while leaving the antisense single-stranded DNA probe intact. After purification by a QIAquick column, the exposed sticky ends are blocked by the use of Klenow exo⁻ and dideoxyribonucleotides (ddNTP's). This manipulation alters the sticky ends. Moreover, it prevents the ddNTP-blocked DNAs from sequence extension due to the lack of free 3′-OH group in ddNTP's. The blockage may be further enhanced by adding terminal deoxyribonucleotide transferase (TdT), which incorporates ddNTP's randomly to the 3′ end of any DNA without the need of a template.

Probe Hybridization, ddNTP Blockage, RNase Digestion, and DSE—After denaturation at 70° C. for 30 minutes, the T7 transcription products are subjected to hybridization with the antisense wild-type DNA probe in 10 mM Tris pH 7.5, 1.25 M NaCl, and 5 mM EDTA at 70° C. for 1 hour, followed by sequence blockage with Klenow exo⁻, TdT and ddNTP's at 37° C. for 4 hours. The ddNTP blockage after the formation of RNA:DNA heteroduplexes prevents partially transcribed RNAs and any free 3′-OH from sequence extension in later steps. After purification with a Qiaquick spin column, the resulting RNA:DNA heteroduplexes are digested with 10 units of RNase ONE in an optimized buffer (Promega, Madison, Wis.), followed by sequence extension in a reaction cocktail containing 5 units of Klenow enzyme, 5 units of Taq DNA polymerase, 50 mM NaCl, 5 mM MgCl₂, 250 □M of deoxyribonucleotide mix (dNTP's), and 5 mM DTT at 37□C for 30 minutes, and then at 70□C for another 30 minutes. Following DSE with Klenow/Taq DNA polymerase, the resulting products are digested with Bam HI at 37□C for 4 hours and then heat inactivated at 65□C for 30 minutes.

Adapter Preparation, Ligation, and Formation of Mutant/Adapter Hybrids—Two oligonucleotides, Adp-Bgl(+) 5′-GAGATCTTGCTGCCCGAAACTGCCT-3′ and Adp-Bgl(−) 5′-AGGCAGTTTCGGGCAGCAAGATCTC-3′, are annealed in equimolar at the final concentration of 10 ng per microliter in a buffer containing 50 mM NaCl and 10 mM Tris, pH 8.3 at 65□C for 30 minutes. The resulting double-stranded oligonucleotides are then digested with BglII to create an adapter carrying a 5′ tetranucleotide “GATC” overhang, which is compatible with the sticky end of BamHI digest. Following dephosphorylation, the BglII adapters are then subject to ligation with the BamHI digested DSE products described in the previous paragraph. In the case of the wild type RNA:DNA heteroduplexes, ligation with the adapters does not take place due to the fact that the BamHI sequences are protected from digests. In contrast, the nicks of mutant RNAs by RNase digestion, synthesis of new DNA strands 3′ to the nicks, and the creation of BamHI sticky ends in the mutants permit the formation of mutant/adapter hybrids after ligation with the BglII adapter. S1 nuclease digestion was then performed to degrade free or exposed single-stranded DNA's and RNA's.

PCR Amplification of BCR/abl TK Mutant/Adapter Hybrids—Semi-nested PCR is performed to amplify the BCR/abl mutant/adapter hybrids. Forward primer TKF1(+) 5′-GAGAACCACTTGGTGAAGGT-3′ and reverse primer AdpR(−)5′-AGGCAGTTTCGGGCAGCAAGATC-3′, are used in the first round of PCR. Primer TKF1(+) resides at 76 bp 5′ to the BamHI/BglII ligation site. Primer AdpR (−) shares the same sequences as Adp-Bgl(−) except that AdpR(−) is shorter by two nucleotides at the 3′ end because these two nucleotides are deleted in the mutant/adapter hybrids following the BamHI/BglII ligation. Using forward primer TKF2 (5′-TGAGCAGGTTGATGACAGG-3′), the reverse primer AdpR(−), and a double fluorescence-labeled probe [ABLTKR 5′-(6-FAM)-GCATGGGCTGTGTAGGTGTC-(TAMRA)-3′], the second round of PCR is performed in an ABI 7900HT Sequence Detection System (Applied Biosystems, Foster City, Calif.) in the following cycling conditions: 95□C for 30 seconds, 60□C for 15 seconds, and 72□C for 45 seconds for 45 cycles after initial heat activation of Taq DNA polymerase GOLD for 10 minutes. By means of the cycle threshold method, the quantities of the BCR/abl TK mutants in the tested samples are calculated against the standard curve plots established by a set of serially diluted positive samples (1:10, 1:10², 1:10³, 1:10⁴, 1:10⁵, 1:10⁶) prepared from a patient who carried 100% Ph chromosome and an BCR/abl TK mutation documented by DNA sequencing. The standard curves can also be established by a set of serially diluted positive constructs in different quantities: 10⁸, 10⁷, 10⁶, 10⁵, 10⁴, 10³, 10², 10 copies.

### Example 4

### Detection of Small Numbers of Leukemia-Cells in B-Cell Leukemia Cells Mixed with a Normal Blood Sample

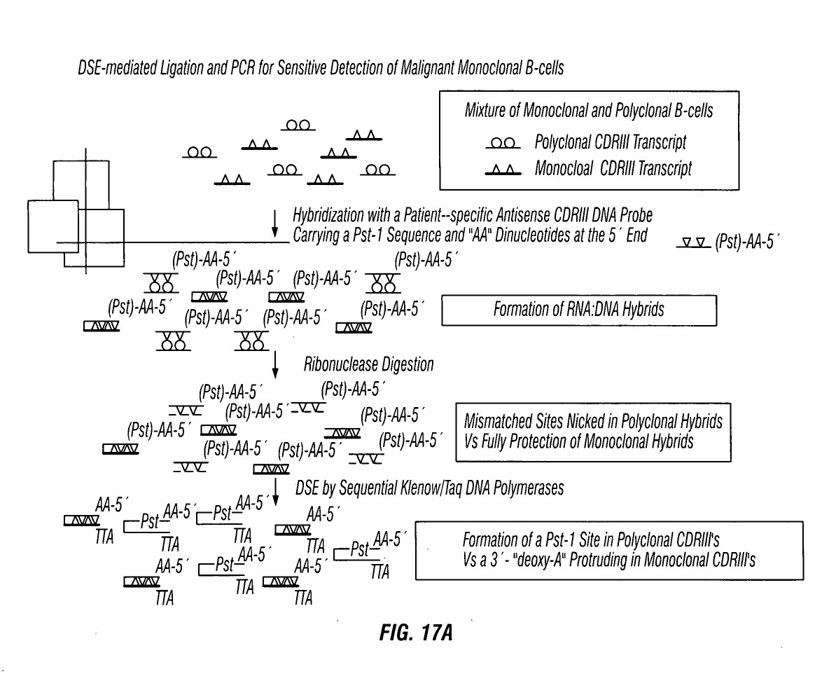

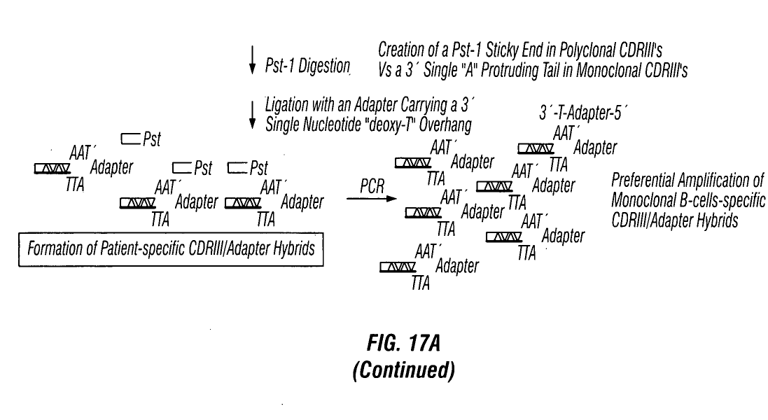

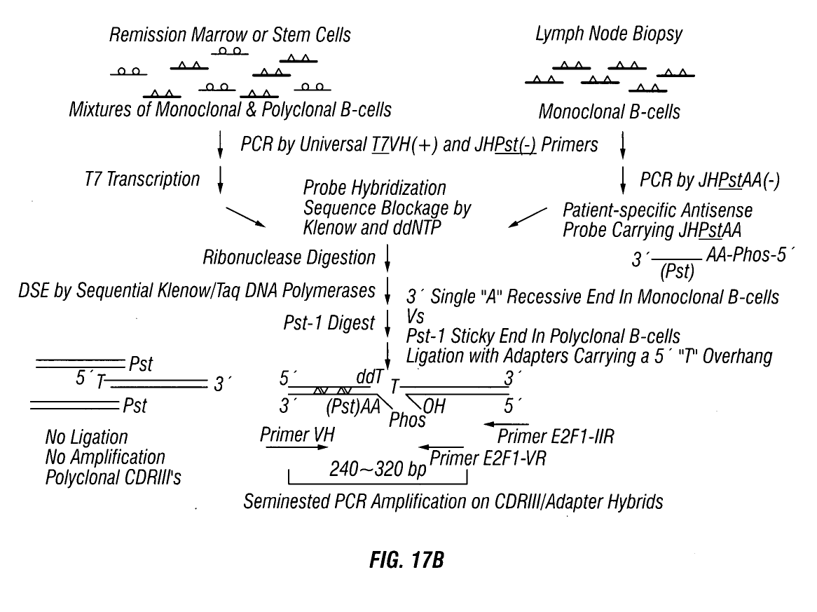

Patients with full-blown B-cell malignancies usually present with abundant monoclonal B-cells that share a uniformly rearranged Ig gene, as opposed to benign polyclonal B-cells that carry hundreds of thousands of different Ig gene rearrangements. When patients achieve remission after treatment, a small number of residual malignant monoclonal B-cells might still be present among hundreds of thousands of polyclonal B-cells. Since the malignant B-cells in each patient have their own unique signature in the CDRIII sequences, the detection of such sequences in remission samples provides solid evidence for the presence of minimal residual disease. FIGS. 17A and 17B are both schematic illustrations for a DSE-applied assay for the detection of a small number of malignant monoclonal B-cells among several hundred thousand polyclonal B-cells.

To study the feasibility and sensitivity of the DSE-applied assay in the detection of monoclonal B-cell-specific CDRIII sequences, the B-cell leukemia cell line B15 in serial dilutions (1:2, 1:10, 1:10², 1:10³, 1:10⁴) and a normal blood sample were examined. The CDRIII regions in both monoclonal B-cells and polyclonal B-cells are amplified from a patient's remission samples and peripheral blood stem cell (PBSC) samples through PCR using a forward primer T7VHF and a reverse primer JHPstR. The sequence of the T7VHF primer is derived from the framework III of the V_{H} region of the Ig heavy chain gene and carries a T7 promoter sequence at its' 5′ end. The JHPstR primer is derived from the consensus sequences at the 3′ end of the J_{H} region with an embedded Pst-1 restriction enzyme site.

The resulting T7 promoter-carrying CDRIII DNA fragments are then subject to RNA transcription with T7 RNA polymerase. Moreover, another primer, JHPstRAA, which carries an “AA” dinucleotide at the 5′ end of JHPstR is synthesized. An antisense CDRIII DNA probe specific for the patient's malignant B-cells from the patient's pretreatment lymph node or bone marrow samples is synthesized by PCR using the B-15 CDRIII fragments as templates and the JHPstRAA primer.

The probe is then hybridized with the CDRIII transcript mixtures amplified from the patient's remission and also the PBSC samples, resulting in either fully matched RNA:DNA heteroduplexes in the case of the patient's monoclonal B-cell-specific CDRIII's, or partially mismatched CDRIII heteroduplexes in the case of polyclonal B-cells. After the formation of RNA:DNA heteroduplexes, Klenow exo- and dideoxyribonucleotides (ddNTPs) are added to block partially transcribed RNA. This manipulation also adds a single nucleotide ddTTP in the fully matched RNA:DNA heteroduplexes that carry a 5′ double-nucleotide “AA”, thus resulting in the formation of a 5′ single-nucleotide “A” overhang.

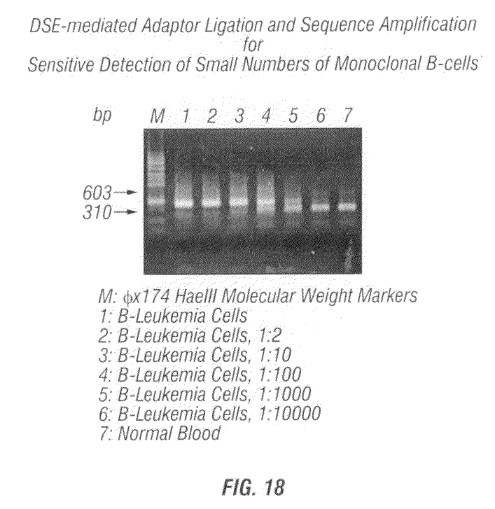

The B-15 CDRIII transcripts are complementary with the antisense probe and thus they are fully protected from RNase ONE digestion. In contrast, the CDRIII transcripts of polyclonal B-cells will be nicked upon treatment with RNase ONE™ due to partial mismatches. Following ribonuclease digestion, the RNA strands of the mismatched heteroduplexes in polyclonal CDRIII's are nicked thus releasing free —OH groups which permit sequence extension by a combination of Klenow enzyme and Taq DNA polymerase. Following DSE with sequential application of Klenow and Taq DNA polymerases, the nicked RNAs of the polyclonal CDRIII heteroduplexes initiate synthesis of new DNA strands to displace the RNA strands 3′ to the nicked sites, thereby forming double stranded DNAs that carry a Pst-1 site near the 3′ end of the J_{H} region. In contrast, the patient's malignant B-cell-specific CDRIII heteroduplexes retain their RNA strands, thereby remaining inert to Pst-1 digestion. Therefore, following DSE with sequential Klenow/Taq DNA polymerases and Pst-1 digestion, two different sticky ends are formed: 1) a 5′ single “A” overhang in the fully matched CDRIII DNA:RNA heteroduplexes of the B-5 cells, versus 2) a Pst-1 sticky end in the polyclonal CDRIII heteroduplexes. After Pst-1 digestion, the polyclonal CDRIII heteroduplexes are cut into two pieces: one small fragment (<20 base pairs) that can be easily eliminated, and the remaining large heteroduplex that carries a Pst-1 sticky end. In contrast, the patient's malignant B-cells-specific CDRIII heteroduplexes carry 5′ single nucleotide “deoxy-A” overhangs on their DNA strands. The single nucleotide overhangs permit ligation with adapters carrying a 5′ “deoxy-T” overhang, but not with the polyclonal CDRIII heteroduplexes that harbor a Pst-1 sticky end. As a result, patient-specific CDRIII/adapter hybrids are formed. The B-15-specific CDRIII/adapter hybrids can be preferentially amplified through PCR using primers derived from the V_{II}, region and the adapter. As shown in FIG. 18, amplicons of expected size (˜240 bp) in different intensities are detected in correlation with the dilutions of B-15 cells, but not in the normal blood sample. Even in the dilution of 1:10⁴, a faint band was detected (lane 6 of FIG. 18).

### Example 5

### Detection and Quantification of Minimal Residual Monoclonal B-Cells by DSE-Applied CDRIII Assay in B-Cell Lymphoma Patients in Clinical Remission

To demonstrate the clinical applicability of the DSE-applied CDRIII assay, we studied six refractory B-cell lymphoma patients who received autologous transplant for salvage therapy. A B-cell leukemia cell line, B15, was used as a positive control and peripheral blood samples from normal donors were used as negative controls. Three types of samples were obtained from each patient: 1) a pretreatment, paraffin-embedded lymph node (LN) biopsy sample, 2) a bone marrow (BM) aspirate obtained when patients achieved clinical remission after salvage chemotherapy, and 3) CD34+ peripheral blood stem cells (PBSC) harvested at 3 to 4 weeks after the remission bone marrow samples were obtained.

DNA Extraction from LN Biopsy Slides—Paraffin-embedded tissue sections of the patients' pretreatment lymph nodes were examined under a light microscope. Those areas consisting of more than 90% lymphoma cells were marked and subject to DNA extraction as the following: deparaffinization with xylene for 5 minutes, washing with 75% ethanol, air drying, dissolved in digestion solution containing 1×SSCE, 10% SDS and 2.5 □g/□L proteinase K and then scraped into a microcentrifuge tube. After incubation at 55□C for 2 hours, the digested samples were incubated at 98□C for 15 minutes to inactivate the proteinase K.

PCR Amplification of the CDRIII Fragments in LN, BM, and PBSC Samples—DNA's extracted from LN, BM and PBSC were subject to PCR to amplify the CDRIII fragments using a forward primer T7VHF, 5′-TTAATACGACTCACTATACGGCCGTATATTACTGT-3′, and a reverse primer JHPstR, 5′-ACCTGCAGGAGACGGTGACC-3′. The sequences of T7VHF were derived from the framework III of the V regions of the Ig heavy chain (IgH) gene and carried T7 promoter sequences at its 5′ end. The sequences of JHPstR were derived from the consensus sequences at the 3′ end of the J regions of the IgH and carried a Pst-1 site. At the end of PCR, the amplicons were purified with a QIAquick PCR purification kit (QIAGEN, Valencia, Calif.).

Preparation of Patient-specific Antisense CDRIII Probes—Using a 5 phosphorylated reverse primer JHPstR-AA, the T7VHF/JHPstR PCR products of the patients' LN samples were used as templates to synthesize patient-specific antisense CDRIII probes. The JHPstR-AA primer carried the JHPstR sequences with a dinucleotide “AA” attached to its 5′ side. After PCR, the amplicons were digested with two frequent cutting restriction enzymes, Hha I and HpaII, to cut double stranded DNA into small pieces and retain the antisense single-stranded DNA probe intact. The antisense CDRIII probes were then purified by a QIAquick PCR purification kit before further use.

Transcription of CDRIII's, Probe Hybridization, RNase Digestion and DSE—RNA transcription was performed using the T7VHF/JHPstR PCR products of the patients' BM and PBSC in a T7 transcription optimized buffer containing 750 □M of ribonucleotide mix, 5 mM of dithiothreitol, 40 units of T7 RNA polymerase, and 20 units of RNasin at 37□C for 2 hours and then digested with RNase free DNase for 3 hours. After denaturation at 70□C for 30 minutes, the T7 transcription products were subjected to hybridization with their corresponding antisense CDRIII probes in 10 mM Tris pH 7.5, 1.25 M NaCl, and 5 mM EDTA at 70□C for 1 hour, followed by sequence blockage with Kienow exo⁻ and ddNTP and then purified by a QuiaQuick spin column. After digestion with 10 units of RNase ONE (Promega, Madison, Wis.), sequence extension was then performed in a reaction cocktail containing 5 units of Klenow enzyme, 5 units of Taq DNA polymerase, 50 mM NaCl, 5 mM MgCl₂, 250 □M dNTP mix, and 5 mM dithiothreitol at 37□C for 30 minutes, and then at 70□ C. for another 30 minutes. After cooling down to 37□C, 10 units of Pst-1 were added and the samples were incubated for 4 hours and then purified with a QIAquick column.

Preparation and Ligation of an Adapter Carrying a 5′ Single nucleotide “T” Protruding End—A PCR fragment spanning the nucleotide positions 525˜814 of the human E2 F1 cDNA was amplified and then digested with Sal-1, resulting in a 62 bp fragment carrying a 3′ recessive end and a 228 bp fragment carrying a 5′ “TCAG” tetranucleotide protruding end. The 228 bp fragment was then partially filled in with Klenow enzyme and a mixture of dTTP, dCTP and dGTP. As a result, an adapter carrying a 5′ protruding “T” was formed. The adapter was then ligated with the DSE products described above. The ligated products were then treated with S1 nuclease to degrade free single stranded DNA's and RNA's.

PCR Amplification of Patient-specific CDRIII/Adapter Hybrids—Semi-nested PCR was performed to amplify patients' monoclonal B-cell-specific CDRIII/adapter hybrids. In the first round of PCR, the forward primer T7VH3F was used along with a reverse primer E2F1-IIR, 5′-AGATATTCATCAGGTGGTC-3′. Then, 250 ng of normal blood DNA were added to the first PCR products and subject to the second round of PCR to amplify the CDRIII/adapter hybrids and a reference target, cyclophilin, using two primer sets: a forward primer T7F, 5′-TTAATAC GACTCACTATA-3′, and a reverse primer E2F1-VR, 5′-ACTGGTGTGGTTCTTGGAC-3′, for the amplification of the CDRIII/adaptor hybrids and a forward primer CyF, 5′-TGAGACAGCA GATAGAGCCAA-3′, and a reverse primer CyR, 5′-TCCCTGCCAATTTGACATCTTC-3′, for the amplification of cyclophilin. The expected size of the amplicons was 240˜320 bp and 100 bp for the CDRIII/adapter hybrids and cyclophilin, respectively. To quantify the amounts of the CDRIII/adapter hybrids, the second round of PCR was performed in an ABI sequence detector 7900HT using the two sets of primers along with two dually fluorescent dye-labeled probes, E2FP (5′-6-FAM-TGAACTGGGCTGCCGAGGTG-TAMRA-3′) and CyP (5′-VIC-AGCACCAATATTCAGTACACAGCTTAAAGCTATAGGTT-TAMRA-3′), in the following cycling conditions: 95□C for 30 seconds, 60□C for 30 seconds, and 72□C for 45 seconds for 45 cycles.

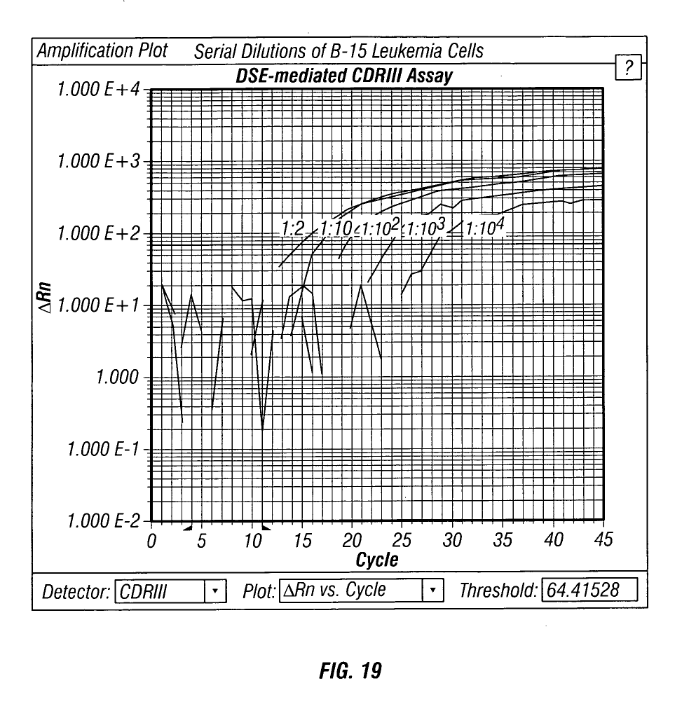

The present example allowed the determination of whether the DSE-applied CDRIII assay could detect small numbers of residual monoclonal B-cells in the remission marrow samples and whether CD34+ cell harvests could completely deprive the residual monoclonal B-cells for autologous transplantation. As described in the previous section, the CDRIII fragments of these samples were PCR amplified using the primers T7VHF and JHPstR. The resulting PCR products of the lymph node samples were subject to making an antisense CDRIII probe specific for each patient using Primer JHPstRAA. The T7VHF/JHPstR PCR products of the remission marrow samples and their corresponding stem cell harvests were subject to transcription with T7 RNA polymerases. Following probe hybridization, blocking with Klenow exo- and ddNTP, RNase ONE digestion, DSE with sequential Klenow/Taq DNA polymerases, Pst-1 digestion, and adapter ligation, semi-nested PCR was performed on the remission marrow samples and their corresponding CD34+ stem cells using primers derived from the V_{H} region and the adapter. To quantify the amounts of residual monoclonal B-cells, the second round of PCR was carried out in an ABI 7900HT Sequence Detector in which the CDRIII/adapter hybrids and an internal standard, cyclophyllin, were co-amplified using their corresponding primers and dually fluorescent dye-labeled probes. Shown in FIG. 19 are the amplification plots of the CDRIII/adapter hybrids of the B-15 leukemia positive control cells that were serially diluted with a normal blood sample at the ratios of 1:1, 1:10, 1:10², 1:10³, and 1:10⁴. Shown in FIG. 20 are the amplification plots of the remission bone marrow (Left Panel) and the peripheral blood stem cells (Right Panel) of a representative patient in whom residual monoclonal B-cells were detected in the remission marrow by the DSE-applied CDRIII assay and the corresponding peripheral blood stem cells appeared negative.

Of the six patients, two were negative in BM's and PBSC's. In the remaining four, their BM's were positive and quantified by the cycle threshold method as 1.7×10⁻¹, 9.5×10⁻³, 2×10⁻⁷, and 6.8×10⁻⁶, respectively. Their corresponding PBSC's were determined as 2.9×10⁻⁶, 1.5×10⁻⁵, 0, and 0, respectively.

To verify these results, the PCR products were subject to size fractionation by gel electrophoresis. Shown in FIG. 21 are the results of the paired remission marrow and peripheral blood stem cell samples in two representative patients (Lanes 4-7) along with three serially diluted positive controls at 1:100, 1:1,000 and 1:10,000, respectively (Lanes 1-3).

### Example 6

### DSE Assay for Microsatellite Polymorphism Analysis

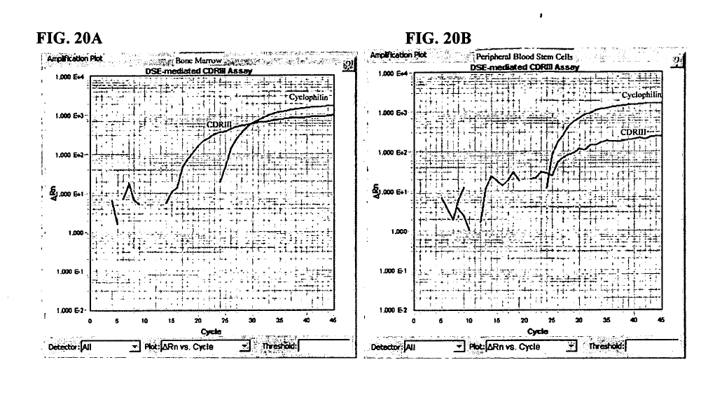

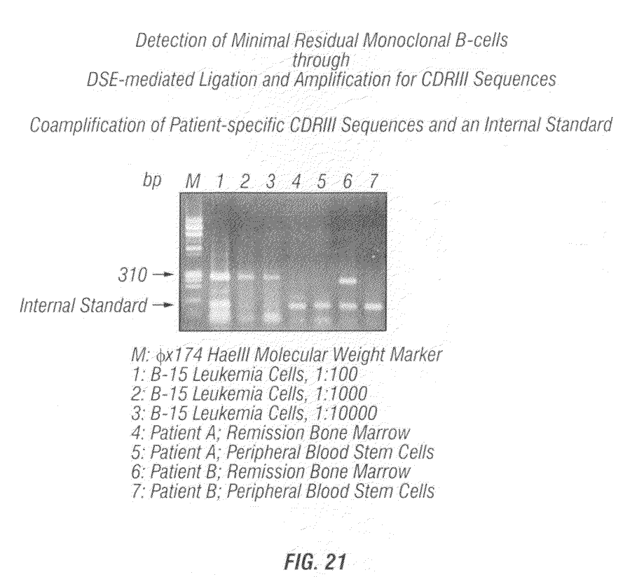

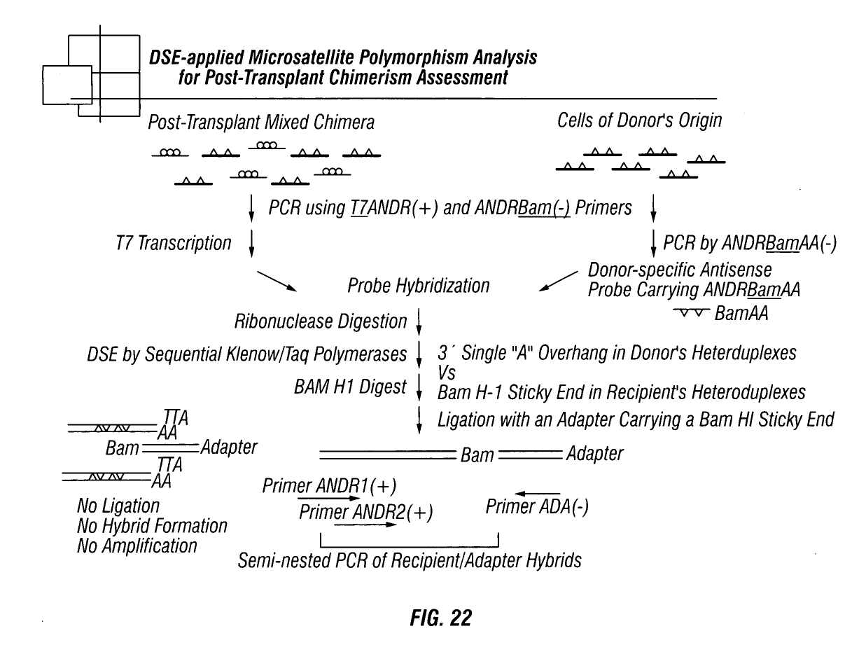

To study the feasibility and sensitivity of the DSE-applied strategy in the identification of cell origin by means of microsatellite polymorphism markers, two different DNA samples, designated as “A” and “B”, that showed different patterns in the polymorphism site at the 5′ end of the Androgen Receptor (ANDR) gene were used. FIG. 22 is a schematic of the present example.

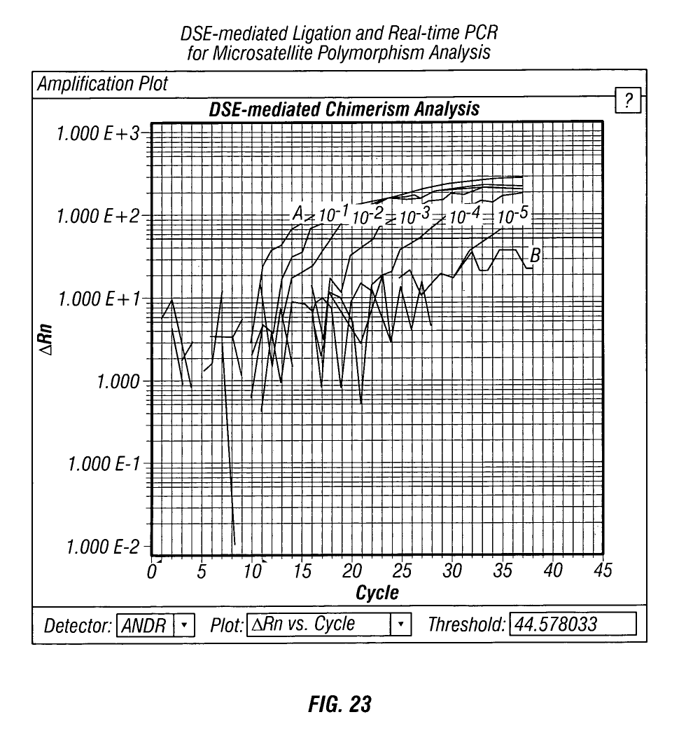

A mixing experiment was performed to form DNA mixtures with different “A” to “B” ratios at 1:10, 1:10², 1:10³, 1:10⁴, and 1:10⁵. The DSE-applied assay began with PCR amplification of the DNA samples using a forward primer, T7ANDR(+), and a reverse primer, ANDRBam(−). The sequences of T7ANDR(+) carried T7 promoter sequences at its 5′ end and the sequences of ANDRBam(−) carried a Bam H1 site. The resulting PCR products were subject to transcription with T7 RNA polymerases and then hybridized with an antisense ANDR DNA probe specific for Sample B. As a result, the transcripts of Sample B's origin were complementary with the antisense probe, thus fully protected from RNase ONE™ digestion. In contrast, the transcripts of Sample A's origin were nicked by RNase ONE due to partial mismatches. Following DSE and Bam-H1 digestion, sticky ends carrying Bam-H1 sequences were formed in the DNA:RNA heteroduplexes of Sample A's origin, but not in the heteroduplexes of Sample B's origin. Consequently, ligation with an adapter carrying a Bam-H1 sticky end resulted in the formation of ANDR/adapter hybrids in the heteroduplexes of Sample A's origin, but not in the heteroduplexes of Sample B's origin. Through semi-nested PCR using primers derived from ANDR and the adapter, the ANDR/adapter hybrids originated from Sample A were preferentially amplified. Shown in FIG. 23 are the real-time PCR amplification plots of Sample A, the serially diluted DNA mixtures with the “A” to “B” ratios at 1:10, 1:10², 1:10³, 1:10⁴, and 1:10⁵, and Sample “B”. Except for Sample B, positive curves of different intensities were detected, and the positives appeared correlated with the dilution factors.

### Example 7

### Sensitive Detection of the Cells of Recipients' Origin in AML Patients Post Allografts—Simultaneous Detection of Recipient and Donor Cells

DSE-mediated Differential Ligation followed by PCR can also be applied to the detection of fully matched and partially mismatched targets at the same time. Chimerism analysis post-allogeneic transplant is an excellent example of this application, i.e., both the recipient's cells and the cells of donor's origin are targets of interest. In this study, the assay is devised for sensitive detection of a small number of recipients' cells as a surrogate indicator for the presence of minimal residual disease or an early evidence of disease recurrence in AML post transplant.

Microsatellite polymorphism assays and SNP analysis are frequently employed to assess the engraftment status and to determine whether there is a mixed chimera post-transplant. However, the sensitivity of the conventional assay is limited to 1-5%. FIG. 22 summarizes a strategy of the DSE-applied assay that permits sensitive detection of a post-transplant mixed chimera at a level that is several hundred times more sensitive than current technologies.

In short, transcripts of a polymorphism marker in post-transplant samples are amplified through a combination of PCR and T7 RNA polymerases, using a forward primer (T7FP) and a reverse primer (RPBam) carrying an embedded Bam H1 restriction enzyme sequence. If Klenow exo⁻ is to be used for DSE, another primer, RP-BamAA(−), is needed. RPBamAA that carries an “AA” dinucleotide attached to the 5′ end of RPBam is used to make a donor-specific, antisense single stranded DNA probe. If DSE is to be performed by the sequential Klenow/Taq approach, primer RP-Bam(−) is sufficient for making a donor-specific, antisense single-stranded DNA probe. Amplified transcripts from post-transplant samples are then hybridized with the donor-specific probe. As a result, fully matched RNA:DNA heteroduplexes in transcripts of the donor's origin and partially mismatched heteroduplexes in transcripts of recipient's origin are formed. Through ribonuclease digestion and DSE by Klenow/Taq DNA polymerases, new DNA strands are synthesized and displace the 3′ region of the nicked transcripts in the heteroduplexes of the recipient's origin, leading to the formation of double-stranded DNAs with a restriction enzyme Bam H1 site. In contrast, due to the absence of newly synthesized DNA strands, the embedded Bam-H1 site in the DNA probe remains protected by its complementary RNA strand in the heteroduplexes of the donor's origin. However, partial sequence extension by Klenow exo⁻ results in a single nucleotide recessive end. In other words, a 5′ “deoxy-A” single nucleotide overhang is created at the DNA strand of the heteroduplexes of the donor's origin. Consequently, digestion with Bam-1 results in the formation of a Bam-H1 sticky end in the heteroduplexes of the recipient's origin as opposed to a 5′ “deoxy-A” single nucleotide overhang for the heteroduplexes of the donor's origin.

The formation of two drastically different sticky ends permits differential ligation with two different species of adapters: one adapter for the heteroduplexes of the donor's origin and the other adapter for the heteroduplexes of the recipient's origin. PCR amplification may then be performed by using primers for the recipient's adapter hybrids and/or the donor's adapter hybrids. If the particular interest is sensitive detection of small numbers of recipient's cells as a surrogate indicator for the presence of minimal residual disease in AML post transplant, then PCR may be performed to preferentially amplify the recipient/adapter hybrids.

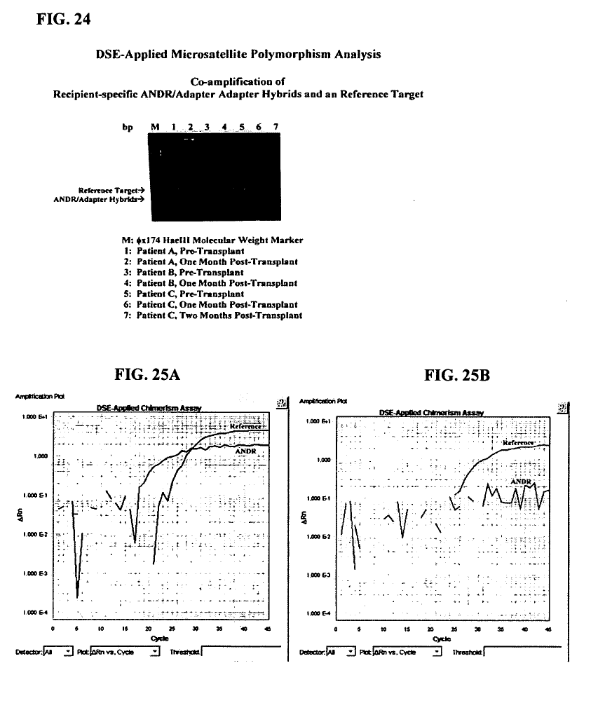

Employing the DSE-applied microsatellite polymorphism assay, five AML patients who received allogeneic stem cell transplantation for salvage therapy and had attained successful engraftment with “100% Donor Cells” as determined by the current standard chimerism analysis were studied. Shown in FIG. 24 are three pre-transplant (Lanes 1, 3, and 5) and four post-transplant blood samples (Lanes 2, 4, 6, and 7) from three representative patients. Residual recipients' cells were detected in one post-transplant blood sample (Lanes #2) while the other three were negative (Lanes 4, 6, and 7).

Real-time PCR was also performed to estimate the quantities of the residual recipients' cells in these seven post-transplant blood samples. Of the seven samples, three were positive and determined as 1.37×10⁻², 1.49×10⁻², and 3.16×10⁻², respectively. The other four were negative for the presence of recipients' cells. Shown in FIG. 25 are the amplification plots of one representative positive sample (Left Panel) and one representative negative sample (Right Panel).

### Example 8

### DSE Assay Using Solid Phase Media

The present example illustrates the DSE mutation assay performed on solid phase media to detect BCR/abl TK mutations using a biotinylated antisense ABL TK probe immobilized on streptavidin magnetic beads. Nine CML patients on Imatinib therapy and three negative control cell lines were used to obtain samples for mutation analysis. Four of the nine patients had Imatinib resistance and respectively carried one of the following four TK mutations documented by DNA sequencing: Y253D, F317L, E255V, and T351I. In the other five patients, DNA sequencing showed no detectable mutation.

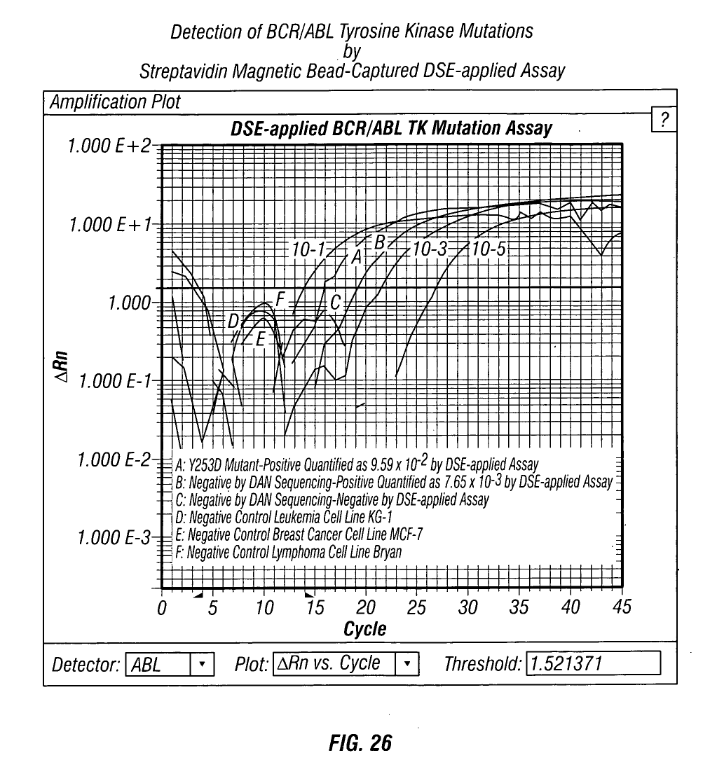

Employing the DSE mutation assay with streptavidin magnetic beads capture, we detected positives in four out of the four mutants. The intensity of the positive as compared to one of our positive control was 9.59×10⁻², 4.89×10⁻¹, 2.76×10⁻¹, 1.71×10^{×1}, respectively. Of the five patients with negative DNA sequencing, two were positive by the present solid phase DSE assay. The positive intensity was estimated as 1.36×10⁻² and 7.65×10⁻³, respectively. The three negative control cell lines were all negative as determined by the solid phase media-captured DSE-applied assay. Shown in FIG. 26 shows the amplification plots of three diluted positive controls (1:10, 1:10³, and 1:10⁵), an Y253D mutant (designated as sample A), a DSE-positive sample that was not detected by DNA sequencing (sample B), a negative sample as determined by both DSE-applied assay and DNA sequencing (sample C), and three negative control cell lines designated as sample D, E, and F, respectively.

The streptavidin magnetic bead-immobilized, DSE-applied BCR/abl TK mutation assay is described below.

### PCR Amplification and T7 Transcription of the BCR/abl TK Domain

As previously described, reverse transcription-mediated semi-nested PCR is performed to amplify the BCR/abl TK domain that carries a T7 promoter sequence at its 5′ end and an embedded BamHI sequence near its 3′ end. Following transcription by T7 RNA polymerase, the resulting TK transcripts are treated with DNase to produce DNA-free, ABL TK transcripts.

### Preparation of a Biotinylated Antisense Single-Stranded Wide-Type DNA Probe for ABL TK

Reverse transcription-mediated PCR is performed with a forward ABL3(+) primer and a reverse ABL7-Bam(−) primer on the total cellular RNA's from a normal individual to amplify a cDNA fragment spanning the ABL TK domain that carries a Bam HI site. The resulting cDNA fragment is then used as a template to synthesize a biotinylated antisense wild type DNA probe by PCR using a biotinylated ABL7-Bam(−) reverse primer. Following PCR, the amplicons are mixed with 25 microliters (˜100 g) of streptavidin magnetic beads (New England Biolab, Ipswich, Mass.) in a microcentrifuge tube and incubated at room temperature for 15 minutes. The immobilized biotinylated ABL TK probe is then denatured in 0.1 M of NaOH at room temperature for 90 seconds. The mixture is then placed onto a magnetic separator rack (New England Biolab, Ipswich, Mass.) to attract the immobilized probe to the side wall of the tube. The tube is then washed twice on the magnetic separator rack with a wash buffer containing 200 mM NaCl, 20 mM Tris, pH 7.4, and 0.1 mM EDTA. After discarding the supernatant, the immobilized single-stranded ABL TK probe is re-suspended in 50 mM NaCl and ready for use.

Probe Hybridization, ddNTP Blockage, RNase Digestion, DSE, Adapter Ligation, and Detection of BAR/ABL TK Mutation by PCR

After denaturation at 70° C. for 30 minutes, the T7 RNA polymerase transcribed ABL TK transcripts are subjected to hybridization with the magnetic bead-immobilized antisense wild-type probe in 10 mM Tris pH 7.5, 1.25 M NaCl, and 5 mM EDTA at 70° C. for 1 hour, followed by sequence blockage with Klenow exo⁻ and ddNTP's at 37° C. for 4 hours. The resulting DNA:RNA heteroduplexes are then washed twice with the wash buffer on the magnetic separator rack. Taken off from the rack, RNase digestion is then performed at 37° C. for 1 hour. After washing on the magnetic separator rack and discarding the supernatant waste, the immobilized, RNase-digested products are subject to sequence extension using the combination of Klenow enzyme and Taq DNA polymerases as previously described. The DSE products are then washed twice on the magnetic separator rack and then digested with Bam HI at 37□C for 4 hours. Because a BamHI site is created by DSE in the presence of TK mutation, the digestion releases the mutant DNA:RNA heteroduplexes into the supernatant. With magnetism on, the supernatant is carefully aspirated from the microcentrifuge tube and then subjected to ligation with a BglII adapter. PCR amplification is then performed to detect BCR/abl TK mutation.

What is claimed is:

1. A method for detecting a genetic mutation comprising the steps of:
incubating at least one sample of heteroduplex molecules comprising a genetic region of interest with a ribonuclease enzyme, wherein said heteroduplex molecules comprise one strand of sense ribonucleic acid (RNA) and one strand of antisense deoxyribonucleic acid (DNA), wherein said sample of heteroduplex molecules comprises a first population of heteroduplexes wherein said RNA and DNA strands are fully hybridized to one another, and a second population of heteroduplexes having at least one member, wherein said RNA strand of said at least one member comprises at least one unhybridized nucleotide within said region of interest, and wherein said ribonuclease cleaves 3′ of said unhybridized nucleotide exposing a 3′ hydroxyl group;
synthesizing a strand of DNA from said 3′ hydroxyl group wherein said antisense DNA is used as a template to produce a sequence extended heteroduplex;
linking a marker to said sequence extended heteroduplex to form a marked heteroduplex; and
detecting said marked heteroduplex.

2. The method of claim 1 further comprising the step of:
sequencing said region of interest of said marked heteroduplex to identify said genetic mutation.

3. The method of claim 1 wherein said marker comprises an adapter DNA molecule.

4. The method of claim 3 wherein said marker comprises a quantifiable molecule, and wherein said detecting step comprises quantifying said marked heteroduplex.

5. The method of claim 3 wherein said detecting step comprises polymerase chain reaction (PCR) amplification of said marked heteroduplex wherein said PCR is carried out using a primer specific for said adapter and a primer specific for said region of interest.

6. The method of claim 5 wherein said marker comprises a quantifiable molecule, and wherein said PCR is real-time PCR.

7. The method of claim 6 wherein said incubating step comprises more than one sample of heteroduplex molecules, wherein each of said samples comprises a unique genetic region of interest.

8. The method of claim 7 wherein said heteroduplex molecule are immobilized on a substrate, wherein each of said more than one samples is in a unique location on said substrate.

8. The method of claim 1 wherein said synthesizing step is carried out by sequential use of two different RNA-primed DNA polymerase enzymes.

9. The method of claim 1 wherein said antisense DNA strand is the wild type sequence of said region of interest.

10. The method of claim 1 wherein said antisense DNA strand is a mutant sequence of said region of interest.

11. The method of claim 1 wherein said DNA strand of said marked heteroduplex is coupled to a immobilizable tag.

12. A method for detecting genetic mutation comprising the steps of:
incubating at least one sample of single stranded RNA together with at least one sample of single stranded antisense DNA to create at least one sample of RNA:DNA heteroduplex molecules comprising a region of interest, wherein said at least one sample of single stranded antisense DNA is immobilized on a substrate, wherein said sample of RNA:DNA heteroduplexes is immobilized and comprises a first population of heteroduplexes wherein said RNA and DNA strands are fully hybridized to one another, and a second population of heteroduplexes having at least one member, wherein said RNA strand of said at least one member comprises at least one unhybridized nucleotide within said region of interest;
incubating said sample of RNA:DNA heteroduplex molecules with a ribonuclease enzyme wherein said ribonuclease cleaves 3′ of said unhybridized nucleotide exposing a 3′ hydroxyl group;
synthesizing a strand of DNA from said 3′ hydroxyl group wherein said antisense DNA is used as a template to produce a sequence extended heteroduplex;
linking a marker to said sequence extended heteroduplex to form a marked heteroduplex; and
detecting said marked heteroduplex.

13. The method of claim 12 further comprising the step of:
sequencing said region of interest of said marked heteroduplex to identify said genetic mutation.

14. The method of claim 12 wherein said marker comprises an adapter DNA molecule.

15. The method of claim 14 wherein said marker comprises a quantifiable molecule, and wherein said detecting step comprises quantifying said marked heteroduplex.

16. The method of claim 15 wherein said incubating step comprises more than one sample of RNA, more than one sample of immobilized DNA and creates more than one sample of heteroduplex molecules, wherein each of said samples of immobilized DNA comprises a unique genetic region of interest, and wherein each of said samples of immobilized DNA is in a unique location on said substrate.

17. The method of claim 12 wherein said synthesizing step is carried out by sequential use of two different RNA-primed DNA polymerase enzymes.

18. The method of claim 12 wherein said antisense DNA strand is the wild type sequence of said region of interest.

19. A kit for detecting genetic mutation comprising:
a multitude of single stranded antisense DNA probes immobilized on a substrate, wherein each of said DNA probes comprises a unique genetic region of interest, and wherein each of said DNA probes is located at a unique location on said substrate; and
a user's guide comprising instructions for executing a method comprising the steps of:
incubating at least one sample of single stranded RNA together with at least one sample of single stranded antisense DNA to create at least one sample of RNA:DNA heteroduplex molecules comprising a region of interest, wherein said sample of RNA:DNA heteroduplexes comprises a first population of heteroduplexes wherein said RNA and DNA strands are fully hybridized to one another, and a second population of heteroduplexes having at least one member, wherein said RNA strand of said at least one member comprises at least one unhybridized nucleotide within said region of interest;
incubating said sample of RNA:DNA heteroduplex molecules with a ribonuclease enzyme wherein said ribonuclease cleaves 3′ of said unhybridized nucleotide exposing a 3′ hydroxyl group;
synthesizing a strand of DNA from said 3′ hydroxyl group wherein said antisense DNA is used as a template to produce a sequence extended heteroduplex;
linking a marker to said sequence extended heteroduplex to form a marked heteroduplex; and
detecting said marked heteroduplex.

20. The kit of claim 19 wherein said multitude of probes target genes selected from the group consisting of oncogenes, tumor suppressor genes, mismatch repair genes, tyrosinc kinase genes, growth factor receptor genes, D-loop and non-D-loop regions of mitochondrial DNA, SNP markers, microsatellite polymorphism markers, and immunoglobulin superfamily genes.

21. An apparatus for detecting genetic mutation comprising:
a reaction chamber comprising at least one removable sample holding device, four walls, a ceiling and a floor, wherein one of said walls comprises a door;
a temperature control element positioned within said reaction chamber said for regulating the temperature of reaction conditions within said reaction chamber,
an electromagnetic member positioned within said reaction chamber that can be turned on to induce magnetism and turned off to remove magnetism,
a fluid dispensing element having access to said reaction chamber for adding and removing reaction materials to samples when samples are present in said reaction chamber, wherein each of said temperature control element, said electromagnetic member, and said fluid dispensing element are movable and may be repositioned to be in proximity with samples when samples are present in said reaction chamber;
a fluorometer coupled with said reaction chamber for detecting any fluorescence present in said chamber.

22. The apparatus of claim wherein said apparatus is automated.
